(19) United States
(12) Patent Application Publication
(10) Pub. No.: US 2023/0272405 A1
(43) Pub. Date: Aug. 31, 2023

(54) FLAVIVIRUS SIGNAL PEPTIDES, VACCINE CONSTRUCTS, AND METHODS THEREFOR

(71) Applicant: University of Connecticut, Farmington, CT (US)

(72) Inventors: Paulo Henrique Verardi, Farmington, CT (US); Brittany Jasperse, Farmington, CT (US); Caitlin M. O’Connell, Farmington, CT (US); Yuxiang Wang, Farmington, CT (US)

(21) Appl. No.: 18/012,143

(22) PCT Filed: Jun. 22, 2021
(86) PCT No.: PCT/US2021/038386

§ 371 Date: Dec. 21, 2022

Related U.S. Application Data
(60) Provisional application No. 63/042,173, filed on Jun. 22, 2020.

(51) Int. Cl. C12N 15/62; C07K 14/005; C12N 15/86; A61K 39/12; A61P 31/14

(52) U.S. Cl. CPC C12N 15/625; A61K 39/12; A61P 31/14; C07K 14/005; C12N 15/86; A61K 2039/5258

(57) ABSTRACT

Disclosed herein are flavivirus signal peptide mutants useful for enhancing the production and secretion of flavivirus envelope (E) viral proteins or virus-like proteins. Also disclosed herein are methods of vaccinating subjects (e.g., human subjects) against a flavivirus comprising administering an expression vector, wherein the expression vector comprises a polynucleotide, and a fusion polypeptide comprising an engineered signal peptide and a flavivirus envelope (E) protein

### CROSS-REFERENCE TO RELATED APPLICATION

This application claims priority to U.S. Provisional Application No. 63/042,173 filed Jun. 22, 2020, which is incorporated herein by reference in its entirety.

### SEQUENCE LISTING

A Sequence Listing, incorporated herein by reference, is submitted in electronic form as an ASCII text file named “8RU9254.txt”, created Jun. 22, 2021 and of size 160 kB.

### GOVERNMENT LICENSE RIGHTS

This invention was made with Government support awarded by the National Institutes of Health, Grant No. R21AI128681. The Government has certain rights in the invention.

### BACKGROUND

The flaviviruses comprise a large family of arthropod borne viruses that cause a diverse array of clinical diseases, including Zika virus (ZIKV), dengue virus (DENV), yellow fever virus (YFV), Powassan virus (POWV), West Nile virus (WNV), tick-borne encephalitis virus (TBEV), and others. A number of other flaviviruses are, and continue to be, recognized as emerging pathogens. Clinical signs differ widely between flavivirus infections, from cardiovascular and hemorrhagic signs to jaundice, neurologic, and teratogenic manifestations.

ZIKV is a rapidly emerging epidemic arboviral disease that has infected over a million people in Brazil. ZIKV has now spread throughout the Americas and to many other countries. While generally an inapparent or mild febrile disease, ZIKV infections have led to thousands of cases of microencephaly in children born to mothers pregnant at the time of infection. There is a growing awareness also of a high rate of Guillian Barré syndrome (GBS) and other neurologic complications following infection, as well as complications leading to thrombocytopenia. Co-endemnicity with DENV may contribute to the disease manifestations and complicates differential diagnosis. In addition, epitope mimics in both viruses may result in a compounded clinical effect.

Accordingly, there is a compelling and urgent need for development of interventions for flaviviruses, such as ZIKV.

### SUMMARY

Engineered signal peptides, fusion proteins comprising the engineered signal peptides an a flavivirus E protein and/or prM protein, expression vectors for the fusion proteins, and methods of using the expression vectors are generally disclosed herein.

In one aspect, disclosed herein are engineered signal peptides comprising the amino acid sequence X₁GAX₂TSVGIV GLLLTTAMA (SEQ ID NO:1) or the amino acid sequence X₁RSGVX₂WTWIFLTMALTMAMAT (SEQ ID NO:27), wherein X₁ is M or absent and X₂ is A, I, L, M, F, H, V, P, G, Y, W, R, or K.

In some embodiments of the signal peptide. X₁ is M and X₂ is W or K.

In some embodiments of the signal peptide, X₁ is M and X₂ is W.

In another aspect, disclosed herein are fusion polypeptides comprising the engineered signal peptides disclosed herein and a flavivirus comprising an envelope (E) protein.

In some embodiments of the fusion polypeptides, the fusion polypeptides further comprise a flavivirus pre-membrane (prM) protein.

In some embodiments of the fusion polypeptides, wherein the N-terminus to C-terminus ordering of the fusion polypeptide is an engineered signal peptide-prM-E.

In some embodiments of the fusion polypeptides, the flavivirus is selected from one or more of Zika virus, dengue virus, yellow fever virus, Powassan virus, West Nile virus, Japanese encephalitis virus, and tick-borne encephalitis virus.

In some embodiments of the fusion polypeptides, the E protein has the amino acid sequence of SEQ ID NO:2.

In some embodiments of the fusion polypeptides, the E protein has the amino acid sequence of SEQ ID NO:29.

In some embodiments of the fusion polypeptides, the prM protein has the amino acid sequence of SEQ ID NO:3.

In some embodiments of the fusion polypeptides, the prM protein has the amino acid sequence of SEQ ID NO:30.

In another aspect, disclosed herein are polynucleotides encoding the fusion polypeptides disclosed herein.

In yet another aspect, disclosed herein are expression vectors comprising a polynucleotide encoding the fusion polypeptide disclosed herein.

In some embodiments of the expression vectors disclosed herein, the expression vectors comprise a recombinant replication-inducible vaccinia virus (vIND). For example, in some such embodiments, the vIND comprises tetracycline operon elements and replicates only in the presence of a tetracycline. In certain embodiments, the vIND comprises the sequence of any one of SEQ ID NOs:32-40.

In another aspect, disclosed herein are pharmaceutical compositions comprising the expression vectors disclosed herein and a pharmaceutically acceptable carrier.

In another aspect, disclosed herein are vaccines comprising the expression vectors disclosed herein and an adjuvant.

Also disclosed herein are methods of vaccinating a subject against a flavivirus infection comprising administering the expression vector disclosed herein, the pharmaceutical compositions disclosed herein, or the vaccines disclosed herein.

In another aspect, disclosed herein are methods of treating a flavivirus infection comprising administering the expression vector disclosed herein, the pharmaceutical compositions disclosed herein, or the vaccines disclosed herein to a subject.

Alternatively, the methods comprise administering the expression vector disclosed herein, the pharmaceutical compositions disclosed herein, or the vaccines disclosed herein to a subject to a subject at risk of contracting a flavivirus infection.

In some of embodiments of the methods, the flavivirus is selected from one or more of Zika virus, dengue virus, yellow fever virus, Powassan virus, West Nile virus, and tick-borne encephalitis virus. In certain embodiments of the methods, the flavivirus is Zika virus. In certain embodiments of the methods, the flavivirus is Powassan virus.

In another aspect, disclosed herein are methods of producing flavivirus envelope (E) protein. In some embodiments, the method comprises introducing the expression vector disclosed herein into a cell; culturing the cell under conditions permitting expression of the fusion protein; and/or isolating the E protein.

In some embodiments of the methods of producing flavivirus envelope (E) protein, the cell is a mammalian, insect, or yeast cell.

In some embodiments of the methods of producing flavivirus envelope (E) protein, the flavivirus is selected from one or more of Zika virus, dengue virus, yellow fever virus, Powassan virus, West Nile virus, and tick-borne encephalitis virus.

In yet another aspect, disclosed herein are methods of producing flavivirus virus-like particles (VLPs). In some embodiments, the method comprises introducing an expression vector disclosed herein into a cell; culturing the cell under conditions permitting expression of the fusion protein and production of virus-like particles (VLPs); and/or isolating the VLPs.

In some embodiments of the methods of producing flavivirus virus-like particles (VLPs), the cell is a mammalian, insect, or yeast cell.

In some embodiments of the methods of producing flavivirus virus-like particles (VLPs), the flavivirus is selected from one or more of Zika virus, dengue virus, yellow fever virus, Powassan virus, West Nile virus, and tick-borne encephalitis virus.

### BRIEF DESCRIPTION OF THE DRAWINGS

The following is a brief description of each of the drawings, which is presented for the purposes of illustrating the exemplary embodiments disclosed herein and not for the purposes of limiting the same.

FIGS. 1 shows that vaccinia viruses (VACVs) that encode ZIKV prM and E (vIND-ZIKVs), secrete E into the supernatant of infected cells, and form VLPs. Panel (a) is a schematic representation of vIND-ZIKV vaccine constructs in the D5R-D6R locus of VACV. Constructs contained the tet repressor gene (tetR) under the control of a strong synthetic early/late promoter (P_{E/L}), the tet operator sequence (O₂) immediately downstream of the natural D6R promoter (P_{D6R}), and the EGFP gene under the control of the natural F17R late promoter (P₁₁). Signal peptide (SP) variant sequences are listed; underlined M represents methionine added to N-terminus and bold amino acids represent mutations from the natural SP sequence or the JEV SP sequence. MGADTSVGIVGLLLTTAMA is SEQ ID NO:5, MGAWTSVGIVGLLLTTAMA is SEQ ID NO:6, MGAKTSVGIVGLLLTTAMA is SEQ ID NO:7, and MGGNEGSIMWLASLAVVIACAGA is SEQ ID NO:4. Panel (b) is a Western blot of Vero cells (lysates or supernatants) infected with five ZIKV vaccine candidates. Bands of approximately 55 kDa were observed. Panel (c) provides representative brightfield and fluorescence images of cells two days after infection with vIND-ZIKV (D4W SP), showing plaque formation in the presence of a representative tetracycline, doxycycline (DOX) (cytopathic effects on multiple green fluorescent EGFP⁺ cells), or abortive infection (single green fluorescent EGFP⁺ cell) in absence of DOX. Panel (d) provides representative transmission electron microscopy images of ZIKV PRVABC59 virions (left) or VLPs secreted into the supernatant of vIND-ZIKV (D4W SP)-infected cells (right) at two different magnifications.

FIGS. 2 shows that vaccinia viruses (VACVs) that encode POWV prM and E (vIND-POWVs), secrete E into the supernatant of infected cells, and form VLPs. Panel (a) is a schematic representation of vIND-POWV vaccine constructs in the D5R-D6R locus of VACV. Constructs contained the tet repressor gene (tetR) under the control of a strong synthetic early/late promoter (P_{E/L}) and the tet operator sequence (O₂) immediately downstream of the natural D6R promoter (P_{D6R}). Signal peptide (SP) variant sequences are listed; underlined M represents methionine added to N-terminus and bold amino acids represent mutations from the natural SP sequence from POWV strain LB or the JEV SP sequence. MRSGVDWTWIFLTMALTMAMAT is SEQ ID NO:24, MRSGVWWTWIFLTMALTMAMAT is SEQ ID NO:25, MRSGVKWTWIFLTMALTMAMAT is SEQ ID NO:26, and MGGNEGSIMWLASLAVVIACAGA is SEQ ID NO:4. Panel (b) is a Western blot of supernatants of Vero cells infected with POWV vaccine candidates with the natural SP, JEV SP, or D4W mutant SP. developed similarly to the ZIKV vaccine candidates. Bands of approximately 55 kDa were observed. Panel (c) shows representative TEM images of POWV VLPs secreted into the supernatant of vIND-POWV (natural SP)-infected Vero cells at two different magnifications.

FIGS. 3 shows that vIMD-ZIKV replicates only in the presence of DOX and is attenuated compared to vIND in vitro and in vivo. Panel (a) shows mean plaque titers of BS-C-1 cells infected with wild-type strain Western Reserve (WR), vIND or vIND-ZIKV (D4W) at an MOI of 5 in the absence or presence of 1 µg/ml DOX. At 0 or 24 h, cells were collected and lysates were titered in duplicate on BS-C-1 cells in the presence of 1 µg/ml DOX. Plaques were counted 2 days post-infection and mean titers of triplicate samples are shown. Panels (b) and (c) are graphs of percent weight of CB6F₁ mice as a function of days after inoculation with vIND or vIND-ZIKV (D4W). CB6F₁ mice (n=5) were inoculated intranasally with 2 × 10⁴ PFU vIND or vIND-ZIKV (D4W) in either the absence (b) or presence (c) of 0.125 mg/ml DOX in the drinking water and were weighed daily for 21 days. Asterisks represent statistical significance (* p<0.05, † p<0.001) by two-way ANOVA (a) or two-way repeated measures ANOVA (b and c). Error bars represent SD.

FIG. 4 is a graph showing vIND-ZIKV vaccination of mice stimulates E-specific IFN-γ-secreting splenocytes (T cells) after one week. Images of representative wells are shown below each group. Antigen-specific IFN-γ-secreting splenocytes in C57BL/6 mice (n=5) vaccinated intramuscularly with either PBS, vIND, or vIND-ZIKV (D4W) at 10⁷ PFU were measured 7 days post vaccination by ELISPOT with E peptide IGVSNRDFVEGMSGG. Data is shown as spot-forming cells (SFC) per 10⁶ splenocytes. Asterisk represents statistical significance (p<0.01) with the Kolmogorov-Smirnov test when comparing to the PBS-vaccinated control group. Horizontal line represents mean and error bars represent SD.

FIGS. 5 presents graphs showing that vIND-ZIKV vaccination elicits E-specific IgG and neutralizing antibodies. Humoral immune responses in C57BL/6 mice (n=8) vaccinated intramuscularly with 10⁷ PFU vIND or vIND-ZIKV (D4W) are shown 4 weeks after vaccination. Panel (a) ZIKV E-specific IgG titers were measured by ELISA at week 4. Panel (b) Plaque reduction neutralization tests (PRNTs) were performed on serum collected from mice vaccinated with vIND or vIND-ZIKV. The PRNT₅₀ was calculated as the reciprocal of the dilution that resulted in at least 50% reduction in ZIKV plaques. Naïve sera (week 0) had PRNT₅₀ titers <4 (data not shown). Statistical significance with the Kolmogorov-Smirnov test is shown as * p<0.01 or † p<0.001. Horizontal lines represent geometric mean and error bars represent SD. LLD is the lower limit of detection.

FIGS. 6 show that single vaccination with vIND-ZIKV is protective in mice against ZIKV challenge. Panel (a) is a schematic representation of the vaccination/challenge schedule and timing of blood collection. Six-week-old immunocompetent C57BL/6 mice (n=8) were vaccinated intramuscularly with PBS or 10⁷ PFU vIND or vIND-ZIKV (D4W) at weeks 0 and 2. Mice were challenged 2 weeks post-boost with 10⁴ PFU ZIKV strain PRVABC59 intraperitoneally, 1 day after being administered 2 mg anti-IFNAR1 antibody intraperitoneally. Panel (b) is a graph showing ZIKV E-specific IgG titers, measured by ELISA, at weeks 0, 2, 4 (1 day prior to challenge), and 6 for each group. Panel (c) is a graph showing PRNTs for each group, performed in 2-fold dilutions on pooled serum, collected for each group at the indicated time points. Panel (d) is a graph of ZIKV NS1-specific IgG titers for each group, measured by ELISA, at weeks 4 (1 day prior to challenge) and 6. Panel (e) is a graph of plaque forming unit (PFU) equivalents/ ml (viremia) for each group measured in serum collected 2 days after ZIKV challenge by qRT-PCR. Statistical significance determined by two-way repeated measures ANOVA (b and d) or unpaired t tests (e) compared to PBS-vaccinated control group is shown in the panels as * p<0.05 or † p<0.005. Horizontal lines represent geometric mean and error bars represent SD. LLD is the lower limit of detection.

FIGS. 7 shows that two vaccinations with vINO-ZIKV are required to protect mice with prior immunity to VACV (vIND) to ZIKV challenge. Panel (a) is a schematic representation of the vaccination/challenge schedule and timing of blood collection in VACV-primed mice. Six-week-old immunocompetent C57BL/6 mice (n=8) were vaccinated intramuscularly with PBS or 10⁷ PFU vIND two weeks prior to first vaccination. Mice were then vaccinated with PBS or 10⁷ PFU vIND or vIND-ZIKV (D4W) at weeks 0 and 2. Mice were challenged 2 weeks post-boost with 10⁴ PFU ZIKV strain PRVABC59 intraperitoneally, 1 day after being administered 2 mg anti-IFNAR1 antibody intraperitoneally. Panel (b) is a graph of titers of anti-vector (VACV) antibodies measured in each group by ELISA in pooled serum collected at weeks -2, 0, and 2. Panel (c) is a graph of ZIKV E-specific IgG titers measured in each group by ELISA at weeks 0, 2, 4 (1 day prior to challenge), and 6. Panel (d) is a graph of PRNTs for each group, performed in 2-fold dilutions on pooled serum collected at the indicated time points. Panel (e) is a graph of ZIKV NS1-specific IgG titers in each group, measured by ELISA at weeks 4 (1 day prior to challenge) and 6. Panel (f) is a graph of PFU equivalents/ ml (viremia) for each group, measured in serum collected 2 days after ZIKV challenge by qRT-PCR. Statistical significance determined by two-way repeated measures ANOVA (c and e) or unpaired t tests (f) compared to PBS-vaccinated control group is shown in the panels as * p<0.05 or † p<0.005. Asterisks represent statistical significance (* p<0.05, † p<0.005) by two-way repeated measures ANOVA (c and e) or unpaired t tests (f) compared to PBS-vaccinated control group. Horizontal lines represent geometric mean and error bars represent SD. LLD, lower limit of detection.

### DETAILED DESCRIPTION

Flaviviruses are a group of related viruses that cause lethal diseases. Members of the Flaviviridae family include viruses such as Zika virus (ZIKV), dengue virus (DENV), yellow fever virus (YFV), Powassan virus (POWV), West Nile virus (WNV), tick-borne encephalitis virus (TBEV), and others.

Flaviviruses are icosahedral and contain a positive-sense single-stranded (ss) ribonucleic acid (RNA) genome about 11 kilobases in length encoding a single polypeptide. During maturation, this polypeptide is cleaved by viral and cellular proteases into three structural proteins (capsid (C), precursor-membrane (prM), and glycoprotein envelope (E)) and several non-structural (NS) proteins (Chambers, T. J., Hahn, C. S., Galler, R. & Rice, C. M. Flavivirus genome organization, expression, and replication. Annual review of microbiology 44, 649-88, doi:10.1146/annurev.mi.44.100190.003245 (1990); Kuno, G. & Chang, G. J. Full-length sequencing and genomic characterization of Bagaza, Kedougou, and Zika viruses. Archives of virology 152, 687-96, doi:10.1007/s00705-006-0903-z (2007)). The precursor-membrane protein is cleaved providing the M protein for virion assembly (Zhang et al., EMBO J 22(11):2604-2613, 2003).

ZIKV is primarily transmitted by bites of infected Aedes mosquitos, but can also be transmitted from mother to fetus, or through sexual contact, breastfeeding or blood transfusion (Song, B. H., Yun, S. I., Woolley, M. & Lee, Y. M. Zika virus: History, epidemiology, transmission, and clinical presentation. Journal of neuroimmunology 308, 50-64, doi:10.1016/j.jneuroim.2017.03.001 (2017)).

ZIKV was first isolated from a sentinel monkey in the Zika forest of Uganda in 1947 (Dick, G. W., Kitchen, S. F. & Haddow, A. J. Zika virus. I. Isolations and serological specificity. Transactions of the Royal Society of Tropical Medicine and Hygiene 46, 509-20 (1952)). The first human case was reported in 1960 in Nigeria, followed by limirws sporadic cases until the 2007 outbreak on Yap Island in Micronesia, during which an estimated 73% of the residents became infected with ZIKV (Duffy, M. R. et al. Zika virus outbreak on Yap Island, Federated States of Micronesia. The New England journal of medicine 360, 2536-43, doi:10.1056/NEJMoa0805715 (2009)). A major epidemic of ZIKV infection occurred in French Polynesia in 2013-2014 with an estimated 19,000 suspected cases of ZIKV (Cao-Lormeau, V. M. et al. Guillain-Barre Syndrome outbreak associated with Zika virus infection in French Polynesia: a case-control study. Lancet 387, 1531-39, doi:10.1016/S0140-6736(16)00562-6 (2016)). In May 2015, authorities in Brazil confirmed autochthonous transmission of ZIKV and within five months, it had spread to 14 states within Brazil (World Health Organization. Zika virus outbreaks in the Americas. Releve epidemiologique hebdomadaire / Section d′hygiene du Secretariat de la Societe des Nations = Weekly epidemiological record / Health Section of the Secretariat of the League of Nations 90, 609-10 (2015). In late 2015, increasing numbers of infants born with microcephaly were reported, prompting the Brazil Ministry of Health to declare a Public Health Emergency of National Importance (Heukelbach, J., Alencar, C. H., Kelvin, A. A., de Oliveira, W. K. & Pamplona de Goes Cavalcanti, L. Zika virus outbreak in Brazil. Journal of infection in developing countries 10, 116-20, doi:10.3855/jidc.8217 (2016)) and the World Health Organization to declare a Public Health Emergency of International Concern from February-November 2016. Once it emerged in Brazil, ZIKV spread rapidly throughout Central and South America, amassing over 170,000 confirmed ZIKV cases across 48 countries and territories (Song et al., 2017).

The rapid spread of ZIKV and its association with neurological diseases necessitated the rapid development of a safe and efficacious vaccine. Since the 2015 outbreak, there has been considerable effort to develop vaccines against ZIKV. Vaccine candidates to date are based on several different platforms, including purified inactivated virus, live-attenuated viruses, DNA, mRNA, protein, peptide, and viral-vectored vaccines (Shan, C., Xie, X. & Shi, P. Y. Zika Virus Vaccine: Progress and Challenges. Cell host & microbe 24, 12-17, doi:10.1016/j.chom.2018.05.021 (2018)). Most flavivirus vaccine candidates are based on the E protein (since E is the target of neutralizing antibodies (Chambers et al., 1990)) or co-expression of prM and E, to lead to the formation of virus-like particles (VLPs) (Mukhopadhyay, S., Kuhn, R. J. & Rossmann, M. G. A structural perspective of the flavivirus life cycle. Nature reviews. Microbiology 3, 13-22, doi:10.1038/nrmicro1067 (2005)).

Therefore, methods of enhancing the expression of flavivirus E protein or production of VLPs are needed for the development of effective vaccines and diagnostics for flaviviruses such as ZIKV, DENV, YFV, POWV, WNV, TBEV, as well as other Flaviviridae.

Currently, only very low levels of envelope (E) are expressed when using the natural signal peptide from ZIKV. Even when a signal peptide derived from a flavivirus known as Japanese encephalitis virus (JEV) is used, levels of expression are still low.

No alternative methods to produce high levels of envelope (E) expression exist for the rapid and inexpensive production of flavivirus vaccines or diagnostics.

Therefore, what is needed are alternative flavivirus signal peptides that provide high levels of envelope (E) expression and secretion.

Engineered signal peptides and fusion polypeptides comprising one of the engineered signal peptides and a flavivirus E protein and optionally a flavivirus pre-membrane (prM) protein are therefore provided herein. Polynucleotides encoding the fusion polypeptides and expression vectors for the fusion polypeptides are also disclosed. Use of the engineered signal peptides in expression or vaccine platforms or vectors to express a flavivirus E protein and/or a flavivirus pre-membrane (prM) protein results in enhanced expression and secretion of flavivirus E protein compared to the expression level resulting from use of the natural flavivirus signal peptide, as well as enhanced formation of virus-like particles (VLPs). A high level of expression of E protein or VLPs is advantageous in reducing the cost of production of vaccines based on E protein or VLPs against flaviviral infection. Further, a high level of E protein or VLP expression by vaccine vectors or other vaccine platforms is required for optimal efficacy of the vaccine.

Also disclosed are pharmaceutical compositions and vaccine compositions. When administered to a subject, these compositions express higher levels of E protein or VLPs that elicit an immune response. The compositions confer protective immunity against one or more flaviviruses and when administered to a subject stimulate an immune response to one or more flaviviruses thereby treating, reducing or preventing flavivirus infection.

Disclosed herein is an engineered signal peptide. The engineered signal peptide comprises the amino acid sequence X₁GAX₂TSVGIV GLLLTTAMA (SEQ ID NO:1) or the amino acid sequence X₁RSGVX₂WTWIFLTMALTMAMAT (SEQ ID NO:27), wherein X₁ is M or absent and X₂ is A, I, L, M, F, H, V, P, G, Y, W, R, or K. In certain embodiments, X₁ is M and X₂ is W or K. In preferred embodiments, X₁ is M and X₂ is W.

Also disclosed is a fusion polypeptide comprising an engineered signal peptide disclosed herein and a flavivirus envelope (E) protein. The fusion polypeptide can further comprise a flavivirus pre-membrane (prM) protein. In a preferred embodiment the fusion polypeptide comprises an N-terminus to C-terminus sequence of engineered signal peptide-prM protein-E protein, configured to permit processing of the fusion polypeptide into prM/M and E. The E protein and the prM protein can be from any flavivirus, and need not be from the same flavivirus. Examples of flaviviruses include a Zika virus (ZIKV), a dengue virus (DENV), a yellow fever virus (YFV), a Powassan virus (POWV), a West Nile virus (WNV), a Japanese encephalitis virus (JEV), and a tick-borne encephalitis virus (TBEV). In certain embodiments the flavivirus can be a Zika virus (ZIKV) or a Powassan virus (POWV). In certain embodiments, the E protein is a ZIKV E protein. The amino acid sequence of the ZIKV E protein can be SEQ ID NO:2, or at least 90% identical to SEQ ID NO:2, or at least 98% identical to SEQ ID NO:2. In certain embodiments, the prM protein is a ZIKV prM protein. The amino acid sequence of the ZIKV prM protein can be SEQ ID NO:3, or at least 90% identical to SEQ ID NO:3, or at least 95% identical to SEQ ID NO:3. In certain embodiments, the E protein is a POWV E protein. The amino acid sequence of the POWV E protein can be SEQ ID NO:29, or at least 90% identical to SEQ ID NO:29, or at least 98% identical to SEQ ID NO:29. In certain embodiments, the prM protein is a POWV prM protein. The amino acid sequence of the POWV prM protein can be SEQ ID NO:30, or at least 90% identical to SEQ ID NO:30, or at least 95% identical to SEQ ID NO:30. Either the E protein or the fusion polypeptide can comprise a label or tag that facilitates its isolation and/or detection. An exemplary tag of this type is a poly-histidine sequence, generally around six histidine residues, that permits isolation of a compound so labeled using nickel chelation. Other labels and tags, such as the FLAG tag (Eastman Kodak, Rochester, NY), are well known and routinely used in the art.

A polynucleotide encoding a fusion polypeptide disclosed herein is also provided.

Further disclosed is an expression vector comprising a polynucleotide encoding a fusion polypeptide disclosed herein.

The term “nucleic acid” or “polynucleotide” includes deoxyribonucleic acid (DNA) molecules and ribonucleic acid (RNA) molecules. A polynucleotide may be single-stranded or double-stranded. Polynucleotides can contain known nucleotide analogs or modified backbone residues or linkages, which are synthetic, naturally occurring, and non-naturally occurring, which have similar binding properties as the reference nucleic acid. Examples of such analogs include, without limitation, phosphorothioates, phosphoramidates, methyl phosphonates, chiral-methyl phosphonates, 2-O-methyl ribonucleotides, peptide-nucleic acids (PNAs). A polynucleotide can be obtained by a suitable method known in the art, including isolation from natural sources, chemical synthesis, or enzymatic synthesis. Nucleotides may be referred to by their commonly accepted single-letter codes.

The terms “polypeptide,” “peptide” and “protein” are used interchangeably herein to refer to a molecule formed from the linking, in a defined order, of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis.

The terms “vector”, “expression vector,” and “expression construct” are used interchangeably and mean a nucleic acid sequence containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. Examples of vectors include a plasmid vector, a cosmid vector, a bacteriophage vector, and a viral vector. Examples of viral vectors include a bacteriophage vector, an adenovirus vector, a retrovirus vector, an adeno-associated virus vector, and a vaccinia virus vector. The vector may be manufactured in various ways known in the art depending on the purpose. An expression vector may include a selection marker for selecting a host cell containing the vector. A “recombinant” vector or expression vector means a vector operably linked to a heterologous nucleotide sequence for the purpose of expression, production, and isolation of a heterologous nucleotide sequence. The heterologous nucleotide sequence can be a nucleotide sequence encoding all or part of an engineered signal peptide or a fusion polypeptide disclosed herein. The recombinant vector may be constructed for use in prokaryotic or eukaryotic host cells or cell-free systems by suitable methods known in the art.

As used herein, “heterologous” means that the sequence or cell originates from a foreign species, or, if from the same species, is substantially modified from its native form in composition and/or genomic locus by deliberate human intervention, or that the sequence is designed de novo without reference to any natural sequence. For example, a promoter operably linked to a heterologous polynucleotide is from a species different from the species from which the polynucleotide was derived, or, if from the same or an analogous species, one or both are substantially modified from their original form and/or genomic locus, or the promoter is not the native promoter for the operably linked polynucleotide. “Heterologous sequences” are those that are not operatively linked or are not contiguous to each other in nature. A “heterologous cell” for expression of a polypeptide or nucleic acid refers to a cell that does not normally express that polypeptide or nucleic acid.

Protein “expression systems” refer to in vivo and in vitro (cell free) systems. Systems for recombinant protein expression typically utilize cells transfected with a DNA expression vector that contains the template. The cells are cultured under conditions such that they translate the desired protein. Expressed proteins are extracted for subsequent purification. In vivo protein expression systems using prokaryotic and eukaryotic cells are well known. Also, some proteins are recovered using denaturants and protein-refolding procedures. In vitro (cell-free) protein expression systems typically use translation-compatible extracts of whole cells or compositions that contain components sufficient for transcription, translation, and optionally post-translational modifications such as RNA polymerase, regulatory protein factors, transcription factors, ribosomes, tRNA cofactors, amino acids and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

Further disclosed herein are host cells comprising an expression vector or a polynucleotide. A suitable host cell can be transformed with at least one of the recombinant vectors or at least one polynucleotide disclosed herein.

The host cell of the vector may be any cell that can be practically utilized by the expression vector. For example, the host cell may be a higher eukaryotic cell, such as a mammalian cell, or a lower eukaryotic cell, such as a yeast cell. Further, the host cell may be a prokaryotic cell, such as a bacterial cell. A prokaryotic host cell may be a Bacillus genus bacterium, such as E. coli JM109, E. coli BL21, E. coli RR1, E. coli LE392, E. coli B. E. coli X 1776, E. coli W3110, Bacillus subtilis, and Bacillus thuringiensis; or an intestinal bacterium, such as Salmonella typhimurium, Serratia marcescens, and various Pseudomonas species. A eukaryotic host cell may be a yeast, an insect cell, a plant cell, or a mammalian cell. Examples of mammalian cells include VERO cells (from monkey kidneys), LLC-MK2 cells (from monkey kidneys), MDBK cells, mouse Sp2/0, CHO (Chinese hamster ovary) K1, CHO DG44, PER.C6, W138, BHK, COS-7, 293, HepG2, Huh7, 3T3, RIN, HeLa, HEK-293, African green monkey BS-C-1 (CCL-26), RK-13, BHK-21, COS-1, HEK293T, Expi293TM,.and a MDCK cell line. Examples of insect cells include Sf9, High Five, Drosophila S2, and mosquito cells such as CCL-125 cells, Aag-2 cells, RML-12 cells, C6/36 cells, C7-10 cells, AP-61 cells, A.t. GRIP-1 cells, A.t. GRIP-2 cells, A.t. GRIP-3 cells, UM-AVE1 cells, Mos.55 cells, SualB cells, 4a-3B cells, Mos.42 cells, MSQ43 cells, LSB-AA695BB cells, NIID-CTR cells, and TRA-171, cells. An example of a plant cell is Nicotiana benthamiana. Examples of a yeast include Saccharomyces cerevisiae and Pichia pastoris.

The polynucleotide or recombinant vector including the polynucleotide may be transferred into the host cell using any suitable method known in the art. For example, when a prokaryotic cell is used as the host cell, the transfer may be performed using a CaCl₂ method or an electroporation method, and when a eukaryotic cell is used as the host cell, the transfer may be performed by microinjection, calcium phosphate precipitation, electroporation, liposome-mediated transfection, LIPOFECTAMINE^{®} (Life Technologies Corporation) transfection, or gene bombardment, but is not limited thereto.

After the expression vector is introduced into the cells, the transfected cells can be cultured under conditions favoring expression of the fusion polypeptide. The culturing conditions can also be ones permitting processing the fusion polypeptide or production of virus-like particles (VLPs) from the expressed fusion polypeptide. The fusion polypeptide or products of the processed fusion polypeptide (e.g., prM protein, E protein, and/or virus-like particles (VLPs)) can be recovered from the culture using standard techniques known in the art.

The term “virus-like particles or VLPs”, as used herein, refers to virus particles that do not contain replicative genetic material but present at their surface an E protein in a repetitive ordered array similar to the virion structure. Typically, VLPs also contain prM and/or M, and E proteins. VLPs may be produced in vitro (Zhang et al., J. Virol. (2011) 30 (8):333). VLPs can also be produced in vivo. To that end, nucleic acid constructs (e.g., DNA or RNA constructs) encoding prM and E proteins can be introduced into a cell of a subject, e.g., a human subject, via methods known in the art, e.g., via use of a viral vector. Any viral vector can be used provided it is able to contain and express both prM and E flavivirus sequences.

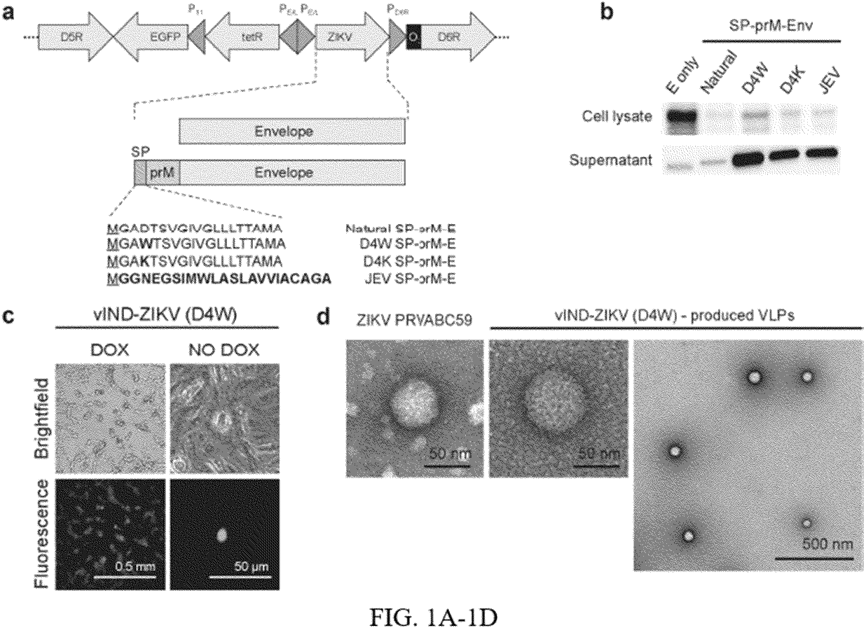

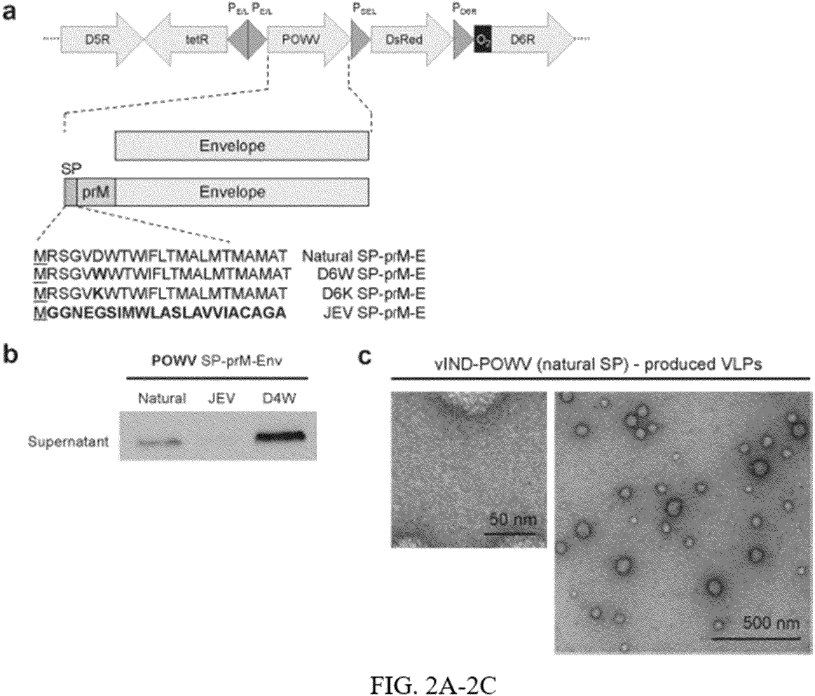

In preferred embodiments, the expression vector is a vaccinia virus (VACV). VACV was used to eradicate smallpox, a disease caused by variola virus, a related poxvirus. VACV has been used as a viral vector for the development of effective human and animal vaccines since it is thermally stable, able to elicit strong humoral and cell-mediated immune responses, easy to propagate, and not oncogenic (Verardi, P. H., Titong, A. & Hagen, C. J. A vaccinia virus renaissance: new vaccine and immunotherapeutic uses after smallpox eradication. Human vaccines & immunotherapeutics 8, 961-970, doi:10.4161/hv.21080 (2012)). However, VACV can cause complications in individuals with conditions such as atopic dermatitis, cardiac disease, and immunosuppression. VACV vectors with a built-in safety mechanism that replicate only in the presence of tetracycline antibiotics have been generated (Hagen, C. J., Titong, A., Sarnoski, E. A. & Verardi, P. H. Antibiotic-dependent expression of early transcription factor subunits leads to stringent control of vaccinia virus replication. Virus research 181, 43-52, doi:10.1016/j.virusres.2013.12.033 (2014); US 20130171189A1). These replication-inducible VACVs (vINDs) contain elements from the tetracycline (tet) operon, specifically the tetR gene encoding the repressor protein (TetR), along with the tetO₂ operator sequence downstream of the promoter of a gene essential for VACV replication. In the absence of tetracyclines, the TetR protein is expressed and binds to the operator sequence, preventing transcription of the essential gene, and consequently replication of the virus. Conversely, in the presence of tetracyclines such as doxycycline (DOX), the TetR protein undergoes a conformational change and no longer binds the operator sequence, allowing transcription of the essential gene and replication of the virus. In the absence of antibiotics, vINDs do not produce infectious progeny and act like other replication-deficient VACV strains such as modified vaccinia Ankara (MVA). Importantly, in the absence of inducer, expression of a fluorescence marker is detected in abortively-infected cells, indicating that even in the absence of viral replication, heterologous antigens are expressed. The vIND-flavivirus constructs disclosed herein are generated by insertion of a flavivirus construct including the regions of the VACV D5R and D6R genes to facilitate homologous recombination with the vaccinia virus, resulting in insertion of the flavivirus construct into the intergenic region between the D5R and D6R genes. Examples of such flavivirus constructs are shown in FIGS. 1a or 2a.

The “percentage of sequence identity” can be calculated by comparing two optimally aligned sequences over the window of comparison, determining the number of positions at which the identical nucleic acid base (e.g., A, T, C, G, U, or I) or amino acid occurs in both sequences to yield the number of matched positions, dividing the number of matched positions by the total number of positions in the window of comparison (i.e., the window size), and multiplying the result by 100 to yield the percentage of sequence identity.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example, the polypeptides GGGGGG and GGGGT have a sequence identity of 4 out of 5 or 80%. For example, the polypeptides GGGPPP and GGGAPPP have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic-F Y W; hydrophobic-A V I L; Charged positive: R K H; Charged negative-D E; Polar-S T N Q. The amino acid groups are also considered conserved substitutions.

“Homology” refers to the percent identity between polynucleotide or polypeptide molecules. Two DNA, or two polypeptide sequences are “substantially homologous” to each other when the sequences exhibit at least about 50%, at least about 75%, at least about 80%, at least about 85%, at least about 90%, at least about 95%, at least about 98% sequence identity over a defined length of the molecules. As used herein, substantially homologous also refers to sequences showing complete identity to the specified DNA or polypeptide sequence.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

A method of vaccinating a subject against a flavivirus infection is also disclosed. The method can comprise administering to a subject an expression vector encoding a fusion polypeptide disclosed herein. The method can alternatively comprise administering to a subject a pharmaceutical composition comprising an expression vector encoding a fusion polypeptide disclosed herein or administering to a subject a vaccine an expression vector encoding a fusion polypeptide disclosed herein.

A method of treating or preventing a flavivirus infection is also disclosed herein. In some embodiments, the method comprises administering to a subject an expression vector encoding a fusion polypeptide disclosed herein. In alternative embodiments, method comprises administering to a subject a pharmaceutical composition comprising an expression vector encoding a fusion polypeptide disclosed herein or administering to a subject a vaccine an expression vector encoding a fusion polypeptide disclosed herein.

A method of reducing risk of contracting a flavivirus infection is also disclosed herein. In some embodiments, the method comprises administering an expression vector disclosed herein, a pharmaceutical composition disclosed herein, or a vaccine disclosed herein to a subject at risk of contracting a flavivirus infection.

In some embodiments of the disclosed methods, the flavivirus is selected from Zika virus, dengue virus, yellow fever virus, Powassan virus, West Nile virus, Japanese encephalitis virus (JEV), tick-borne encephalitis virus, and a combination thereof. In certain embodiments of the disclosed methods, the flavivirus is Zika virus or Powassan virus. In preferred embodiments of the disclosed methods, the flavivirus is Zika virus.

Also disclosed herein are methods of producing flavivirus envelope (E) protein. In some embodiments, the method comprises introducing an expression vector encoding a fusion polypeptide disclosed herein into a cell; culturing the cell under conditions permitting expression of the fusion protein; and isolating the E protein.

Also disclosed herein are methods of producing flavivirus virus-like particles (VLPs). In some embodiments, the method comprises introducing an expression vector encoding a fusion polypeptide disclosed herein into a cell; culturing the cell under conditions permitting expression of the fusion protein and production of virus-like particles (VLPs); and isolating the VLPs.

In some embodiments of the disclosed methods of producing antigens, the cell can be a mammalian, insect, or yeast cell. Examples of such cells are disclosed above.

Any suitable method of culturing the cells can be used. A suitable culture medium and the conditions for the culturing are selected to provide optimal production of the E protein or of VLPs.

In these methods of producing flavivirus antigens, the flavivirus can be selected from one or more of Zika virus, dengue virus, yellow fever virus, Powassan virus, West Nile virus, Japanese encephalitis virus, tick-borne encephalitis virus. In certain embodiments the flavivirus is Zika virus. In certain embodiments, the flavivirus is Powassan virus.

Vaccine candidates for antigens) were generated against ZIKV and POWV. The ZIKV or POWV gene(s) were place under the control of a synthetic vaccinia virus (VACV) early/late (P_{E/L}) promoter to generate replication inducible vaccinia viral vector (vIND) constructs. A schematic of the vaccine constructs are shown in FIGS. 1s and 2a, respectively for ZIKV and POWV.

A first embodiment of a ZIKV vaccine contains the full-length Envelope (E) protein with a methionine added immediately upstream to facilitate translation. A second embodiment of a vaccine includes pre-membrane (prM) and E, along with the putative natural signal peptide (SP) encoded in the last 18 amino acids of capsid (C) protein, to ensure proper folding and secretion of E and to lead to the formation and secretion of VLPs.

A series of ZIKV signal peptide variants were designed that replaced the negatively-charged aspartic acid (D) amino acid (position 4) in the N-terminal region. An N-terminal M residue was optionally added (SEQ ID NO:1). Substitution of the D residue with A, I, L, M, F, V, P, G, Y, W, R, or K, preferably with W or K, more preferably with W, resulted in increased expression of E in the cell lysate when compared to the natural signal protein. A first embodiment of a signal peptide variant replaces the aspartic acid residue with a strongly hydrophobic residue, tryptophan (W), a D4W mutation. A second embodiment of a signal peptide variant replaces the aspartic acid residue with a positively charged lysine (K), a D4K mutation. Minimal secretion of E was detected in the supernatant of cells infected with the vaccine candidate expressing E only (without prM or any signal peptide). Low levels of E were secreted into the supernatant by the natural signal peptide, all as shown in FIG. 1b.

The constructs encoding the desired ZIKV antigens were subcloned into a plasmid. The resulting vectors were transfected into cells infected with a lac-inducible parental virus, and after homologous recombination, vIND-ZIKVs were purified to separate them from parental virus.

The engineered signal peptides significantly enhance expression of E and are useful for improved production of E protein antigens, vaccines and diagnostics for flaviviruses such as Zika virus (ZIKV), dengue virus (DENV), yellow fever virus (YFV), West Nile virus (WNV), tick-borne encephalitis virus (TBEV), and others.

VACVs expressing the prM-Env of the flavivirus Powassan virus (POWV), with the D4W mutation, also showed substantial improvement over the natural or JEV signal peptide (FIG. 1e), thus demonstrating applicability of the engineered signal peptide to flaviviruses other than ZIKV.

When combined with pharmaceutically acceptable carriers, diluents and the like, the E protein antigens, polynucleotides encoding the fusion polypeptides, or expression vectors comprising polynucleotides encoding the fusion polypeptides provide the flavivirus vaccine compositions described herein, which are useful, when administered to a human or animal in an effective amount, for inducing or enhancing an immune response to flavivirus, thus providing a flavivirus vaccine for the treatment, reduction, inhibition or prevention of flavivirus infection.

The exact quantity of a flavivirus vaccine, for example a ZIKV vaccine or a POWV vaccine, to be administered may vary according to the age and the weight of the patient being vaccinated, the frequency of administration as well as the other ingredients (e.g., adjuvants) in the composition. Effective amounts and schedules for administering flavivirus vaccine compositions can be determined empirically. The dosage ranges for administration are those large enough to produce the desired effect in which one or more symptoms of the disease or condition are affected (for example, inhibited, reduced, delayed, or prevented). The dosage should not be so large as to cause substantial adverse side effects, such as unwanted cross-reactions, unwanted cell death, and the like. Generally, the dosage will vary with the type of flavivirus vaccine composition, the species, age, body weight, general health, sex and diet of the subject, the mode and time of administration, rate of excretion, drug combination, and severity of the particular condition. The dosage can be adjusted by the individual physician in the event of any contraindications. Dosages can vary, and can be administered in one or more dose administrations daily.

As used herein, the term “patient,” “individual,” or “subject” refers to a mammal, human or non-human. Non-human mammals include, for example, non-human primates, ovine, bovine, porcine, canine, feline, and murine mammals. In certain embodiments, the patient, individual, or subject is human.

The term effective amount, as used throughout, is defined as any amount, for example, an amount of a vaccine composition, necessary to produce one or more desired immune responses, such as treatment, reduction, or prevention of flavivirus infection. For example, the dosage is optionally less than about 10 mg/kg and can be less than about 9.5 mg/kg, less than about 9 mg/kg, less than about 8.5 mg/kg, less than about 8 mg/kg, less than about 7.5 mg/kg, less than about 7 mg/kg, less than about 6.5 mg/kg, less than about 6 mg/kg, less than about 5.5 mg/kg, less than about 5 mg/kg, less than about 4.5 mg/kg, less than about 4 mg/kg, less than about 3.5 mg/kg, less than about 3 mg/kg, less than about 2.5 mg/kg, less than about 2 mg/kg, less than about 1.5 mg/kg, less than about 1.25 mg/kg, less than about 1.0 mg/kg, less than about 0.9 mg/kg, less than about 0.8 mg/kg, less than about 0.7 mg/kg, less than about 0.6 mg/kg, less than about 0.5 mg/kg, less than about 0.4 mg/kg, less than about 0.3 mg/kg, less than about 0.2 mg/kg, less than about 0.1 mg/kg, or any dosage in between these amounts. The terms “about” or “approximately” are used herein to indicate that a value includes the inherent variation of error for the device, the method being employed to determine the value, or simply error-tolerance of a value. For example, the terms “about” or “approximately” may mean ±1%, ±5%, ±10%, ±15% or ±20% variation from a predetermined value. The dosage can be about 0.1 mg/kg to about 10 mg/kg, about 0.1 mg/kg to about 9 mg/kg, about 0.1 mg/kg to about 8 mg/kg, about 0.1 mg/kg to about 7 mg/kg, about 0.1 mg/kg to about 6 mg/kg, about 0.1 mg/kg to about 5 mg/kg, about 0.1 mg/kg to about 4 mg/kg, about 0.1 mg/kg to about 3 mg/kg, about 0.1 mg/kg to about 2.5 mg.kg, about 0.1 mg/kg to about 2 mg/kg, about 0.1 mg/kg to about 1.5 mg/kg, about 0.1 mg/kg to about 1 mg/kg, or about 0.1 mg/kg to about 0.5 mg/kg. The dosages can be adjusted based on specific characteristics of the vaccine composition and the subject receiving it.

Alternatively, dosage of a replication-defective vaccinia virus expressing flavivirus antigens, for example, E protein or flavivirus-like particles, into a mammalian subject can be expressed as plaque forming units (PFU)/kilogram(kg) subject weight. For example, an effective amount of the vIND-ZIKV can be about 10³ PFU/kg to about 10⁸ PFU/kg. Yet another means to express dosage of the replication-defective vaccinia virus expressing flavivirus-like particles, e.g., ZIKV-like particles, is cell culture infectious dose 50% (CCID50): the amount of a virus sufficient to cause a cytopathic effect in 50% of inoculated replicate cell cultures, as determined in an end-point dilution assay in monolayer cell cultures. The quantity of a vaccinia vector comprised in a vaccine composition of the present disclosure lies within a range of about 10³ CCID50 to about 10⁷ CCID50, about 10³ CCID50 to about 10⁶ CCID50, or about 10³ CCID50 to about 10⁷ CCID50, for example bout 5 × 10³ CCID50 to about 5 × 10⁵ CCID50, for example, about 1 × 10⁴ CCID50 to about 1 × 10⁵ CCID50, for about 10⁵ CCID50.

Generally, the quantity of a VLP within a vaccine composition lies within a range of about 100 ng pg to about 100 pg of VLP, preferably within a range of about 100 ng pg to about 50 pg, preferably within a range of about 100 ng pg to about 20 pg, preferably about 1 pg to about 10 pg. The amount of VLP can be determined by ELISA.

The ability of a vaccine composition disclosed herein to provoke an immune response in a subject (i.e., induce the production of neutralizing antibodies) can be assessed, for example, by measuring the neutralizing antibody titer raised against the flavivirus serotype(s), for example a ZIKV serotype, comprised within the composition. The neutralizing antibody titer may be measured by the Plaque Reduction Neutralization Test (PRNTso) test. Briefly, neutralizing antibody titer is measured in sera collected from vaccinated subjects at least 28 days following administration of a vaccine composition of the present disclosure. Serial, two-fold dilutions of sera (previously heat- inactivated) are mixed with a constant challenge-dose of Zika virus as appropriate (expressed as PFlJ/mL). The mixtures are inoculated into wells of a microplate with confluent Vero cell monolayers. After adsorption, cell monolayers are incubated for a few days. The presence of Zika virus infected cells is indicated by the formation of infected foci and a reduction in virus infectivity due to the presence of neutralizing antibodies in the serum samples can thus be detected. The reported value (end point neutralization titer) represents the highest dilution of serum at which >50 % of Zika challenge virus (in foci counts) is neutralized when compared to the mean viral focus count in the negative control wells (which represents the 100% virus load). The end point neutralization titers are presented as continuous values. As PRNT tests may slightly vary from a laboratory to another, the LLOQ may also slightly vary. Accordingly, in a general manner, it is considered that seroconversion occurs when the titer is superior or equal to the LLOQ of the test.

A vaccine composition according to the present disclosure may be administered in a single dose. A vaccine composition according to the present disclosure may be administered in multiple doses. Doses of a vaccine composition according to the present disclosure may be administered in an initial vaccination regimen followed by booster vaccinations. For example, a vaccine composition according to the present disclosure may be administered in one dose, two doses, three doses, or more than three doses (e.g., four or more doses).

In some embodiments, the first dose and the third dose are administered approximately twelve months apart. For example, an initial vaccination regimen is administered in three doses, wherein the first and third doses of said vaccination regimen are to be administered approximately twelve months apart.

In some embodiments, the vaccine composition disclosed herein is in a first dose, a second dose, and a third dose. In some such embodiments, said first dose and said third dose may be administered approximately twelve months apart. For instance, the vaccine composition is administered in a first dose, a second dose, and a third dose, wherein said second dose is administered about six months after said first dose, and wherein said third dose is to be administered about twelve months after said first dose. Alternatively, the three doses may be administered at zero months, at about three to four months (e.g., at about three-and-a-half months), and at about twelve months (i.e., a dosing regimen wherein the second dose of the composition is administered at about three-and-a-half months after the first dose, and wherein the third dose of the composition is administered at about twelve months after the first dose).

A vaccine composition comprising expression vectors expressing a flavivirus antigen, for example a replication-defective vaccinia virus expressing Zika virus antigens or a replication-defective vaccinia virus expressing POWV antigens, may also comprise one or more adjuvants to enhance the immunogenicity of the flavivirus antigen. In some embodiments, an adjuvant is used in a vaccine composition that comprises an inactivated virus or a VLP or a flavivirus structural protein. An adjuvant also can be used in a vaccine composition comprising a live attenuated virus where the adjuvant does not influence replication.

Suitable adjuvants include an aluminum salt such as aluminum hydroxide gel, aluminum phosphate or alum, but may also be a salt of calcium, magnesium, iron, or zinc. Further suitable adjuvants include an insoluble suspension of acylated tyrosine or acylated sugars, cationically or anionically derivatized saccharides, or polyphosphazenes. Alternatively, the adjuvant may be an oil-in-water emulsion adjuvant as well as combinations of oil-in-water emulsions and other active agents. Other oil emulsion adjuvants have been described, such as water-in-oil emulsions. Examples of such adjuvants include MF59, AF03 (WO 2007/006939), AF04 (WO 2007/080308), AF05, AF06 and derivatives thereof. The adjuvant may also be a saponin, lipid A or a derivative thereof, an immunostimulatory oligonucleotide, an alkyl glucosamide phosphate, an oil in water emulsion or combinations thereof. Examples of saponins include Quil A and purified fragments thereof such as QS7 and QS21. Those skilled in the art will be able to select an adjuvant that is appropriate in the context of this disclosure.

The vaccine compositions disclosed herein are suitably formulated to be compatible with the intended route of administration. Examples of suitable routes of administration include, for instance, intramuscular, transcutaneous, subcutaneous, intranasal, oral, and intradermal. In some embodiments, the preferred route of administration is subcutaneous.

The disclosed expression vectors expressing a flavivirus antigen, for example, a replication-defective vaccinia virus expressing ZIKV antigens, can be provided as a pharmaceutical composition. These include, for example, a pharmaceutical composition comprising an expression vector comprising a polynucleotide encoding a fusion polypeptide disclosed herein and a pharmaceutical carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but are not limited to, saline, buffered saline, artificial cerebral spinal fluid, dextrose, and water.

Depending on the intended mode of administration, the pharmaceutical compositions disclosed herein can be in the form of solid, semi-solid, or liquid dosage forms, such as, for example, tablets, suppositories, pills, capsules, powders, liquids, or suspensions. In some embodiments, the pharmaceutical compositions disclosed herein is in a unit dosage form suitable for single administration of a precise dosage.

In some embodiments, the pharmaceutical compositions disclosed herein include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable that can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material known in the art for use in pharmaceutical formulations. The choice of a carrier for use in a pharmaceutical composition will depend upon the intended route of administration for the composition. The preparation of pharmaceutically acceptable carriers and formulations containing these materials is described in various sources and manuals. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN^{®} (ICI. Inc.; Bridgewater, New Jersey), polyethylene glycol (PEG), and PLURONICS™ (BASF; Florham Park, NJ).

Pharmaceutical compositions or vaccine compositions containing the agent(s) described herein suitable for parenteral injection may include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the flavivirus vaccine compositions disclosed herein include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also include buffering agents.

Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the flavivirus vaccine compositions include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

When used in the methods according to the embodiments of the present invention, flavivirus vaccine compositions can be administered in a number of ways depending on whether local or systemic treatment is desired, and on the area to be treated. The compositions are administered via any of several routes of administration, including orally, parenterally, intravenously, intraperitoneally, intracranially, intraspinally, intrathecally, intraventricularly, intramuscularly, subcutaneously, intracavity or transdermally. Pharmaceutical compositions can also be delivered locally to the area in need of treatment, for example by topical application or local injection. Effective doses for any of the administration methods described herein can be extrapolated from dose-response curves derived from in vitro or animal model test systems.

Throughout, treat, treating, and treatment refer to a method of reducing, inhibiting, preventing, or delaying one or more effects or symptoms of flavivirus infection. The effect of the administration to the subject can have the effect of but is not limited to reducing one or more symptoms associated with flavivirus infection. The effect of the administration to the subject can have the effect of but is not limited to reducing viral load and/or amount. For example, a disclosed method is considered to be a treatment if there is about a 10% reduction in flavivirus infection when compared to the subject prior to treatment or when compared to a control subject or control value. Thus, the reduction can be about a 10% reduction, about a 20% reduction, about a 30% reduction, about a 40% reduction, about a 50% reduction, about a 60% reduction, about a 70% reduction, about a 80% reduction, about a 90% reduction, about a 100%% reduction, or any amount of reduction in between.

As used herein, the terms “prevent, preventing, or prevention” are intended to mean precluding, delaying, averting, obviating, forestalling, stopping, or hindering the onset, incidence, severity, or recurrence of flavivirus infection. For example, the disclosed methods are considered to be a prevention if there is a reduction or delay in onset, incidence, severity, or recurrence of flavivirus infection or associated conditions in a subject susceptible to flavivirus infection as compared to control subjects susceptible to flavivirus infection that did not receive a flavivirus vaccine composition. Several vaccine constructs against ZIKV or against POWV were generated as described herein. A schematic representation of the ZIKV vaccine constructs is shown in FIG. 1a. The ZIKV gene(s) were placed under the control of a synthetic vaccinia virus (VACV) promoter and inserted between VACV genes D5R and D6R by homologous recombination, generating an inducible VACV (vIND) that replicates only in the presence of tetracyclines. Enhanced green fluorescence protein (EGFP) was included in the recombinant VACVs (rVACVs) to expedite purification. The first vaccine candidate contained the full-length Envelope (E) protein with a methionine added immediately upstream to facilitate translation. A second vaccine candidate was designed that included pre-membrane (prM) and E, along with the putative natural signal peptide (SP) encoded in the last 18 amino acids of capsid (C) protein, to ensure proper folding and secretion of E and lead to the formation of virus-like proteins (VLPs).

SPs characteristically contain three distinct domains: an N-terminal (n) region often containing positively-charged residues, a hydrophobic (h) region of at least six hydrophobic residues, and a polar uncharged C-terminal (c) region to facilitate translocation into the endoplasmic reticulum (ER) and in the case of ZIKV capsid SP, to direct prM into the ER lumen for proper secretion of E. Upon reviewing the natural ZIKV SP sequence, the negatively-charged aspartic acid (D) in the n-region (FIG. 1a) was identified as a site that could lead to sub-optimal secretion of E. Therefore, a series of SP variants were designed using TargetP 1.1 software to evaluate the localization of proteins based on the SP sequence. The first variant generated replaced the aspartic acid residue with a strongly hydrophobic residue, of which, tryptophan (W) resulted in the highest secretory pathway prediction score (0.930), compared to the natural SP (score 0.865). An SP variant that replaced the aspartic acid with a positively charged lysine (K) (score 0.878) was also generated. Lastly, a vaccine candidate was generated that included the last 22 amino acids of the SP of Japanese Encephalitis Virus (JEV, score 0.931), on the basis that this sequence was used to target proteins for secretion.

Once constructs containing the desired ZIKV antigens were generated, they were subcloned into a plasmid containing elements of the tetracycline (tet) operon to facilitate generation of vINDs expressing the ZIKV antigens (vIND-ZIKVs). The resulting shuttle vectors were transfected into cells infected with a lac-inducible parental virus, and after homologous recombination, vIND-ZIKVs were purified away from parental virus.

The five ZIKV vaccine candidates (shown in FIG. 1a) were then evaluated for expression of E in both the lysate and supernatant of infected Vero cells by western blot (FIG. 1b). A protein matching the expected size of E (~55 kDa) was observed in the cell lysate for all vaccine constructs, albeit to different levels. Substitution of the D residue with W or K increased expression of E in the cell lysate compared to the natural SP and the JEV SP. As expected, minimal secretion of E was detected in the supernatant of cells infected with the vaccine candidate expressing E only (without prM). Low levels of E were secreted into the supernatant by the natural SP, but those levels increased dramatically when the natural SP was replaced with each of the 3 variants.

Four vaccine candidates for POWV (another flavivirus) were constructed in the same way as described in FIG. 1a. A schematic representation of the POWV vaccine constructs is shown in FIG. 2a. POWV vaccine candidates were evaluated for expression of E in supernatant of infected Vero cells by western blot (FIG. 2B). A protein matching the expected size of E (~55 kDa) was observed in the cell lysate for all vaccine constructs, albeit to different levels. Substitution of the D residue with W increased expression of E in the cell lysate substantially compared to the natural SP or JEV SP (that actually resulted in lower levels of expression). FIG. 2C are transmission electron microscopy (TEM) images of VLPs generated using a POWV vaccine construct disclosed herein

The following are provided for exemplification purposes only and are not intended to limit the scope of the invention described in broad terms above. All references cited in this disclosure are incorporated herein by reference.

### EXAMPLES

### Example 1. Single Dose of Replication-Defective Vaccinia Virus Expressing Zika Virus-Like Particles is Protective in Mice

Several ZIKV vaccine candidates were generated in a vlND backbone to express ZIKV E alone or as VLPs. The vaccine constructs are depicted schematically in FIG. 1a. It was discovered that a novel mutation (D4W) in the natural signal peptide (SP) of prM resulted in increased expression and secretion of E. This vIND-ZIKV was chosen to continue into downstream studies. A single dose of vIND-ZIKV induced robust cell-mediated and humoral immune responses in mice that protected transiently-susceptible C57BL/6 mice from viremia after ZIKV challenge. The vaccine was also tested in the context of prior VACV immunity and it was found that mice previously inoculated with vIND required two doses of vIND-ZIKV to generate high anti-E antibody titers and protect against ZIKV replication as determined by viral loads in blood (viremia).

### Materials and Methods

### Cells

African green monkey BS-C-1 (CCL-26) and Vero (CCL-81) cells were obtained from the American Type Culture Collection (ATCC, Rockville, MD) and were grown in Dulbecco’s modified Eagle medium (D-MEM: Life Technologies, Gaithersburg, MD) supplemented with 5-10% tetracycline-tested fetal bovine serum (FBS, Atlanta Biologicals, Flowery Branch, GA). All cells were grown at 37° C. in 5% CO₂.

### Viruses, Antibodies and Peptides

The L-variant of V ACV strain Western Reserve (WR) was obtained from ATCC (VR-2035) and a clone (9.2.4.8) derived by sequential plaque purification (Grigg, P., Titong, A., Jones, L. A., Yilma, T. D. & Verardi, P. H. Proceedings of the National Academy of Sciences of the United States of America 110, 15407-15412, doi:10.1073/pnas.1314483110 (2013)) was used to generate the recombinant viruses in this study. ZIKV strain PRVABC59 (Asian lineage) was obtained from BEI Resources (National Institute of Allergy and Infectious Disease, National Institutes of Health, USA; NR-50240) and was thawed once and divided into aliquots that were stored at -80° C. New aliquots were thawed for each assay and discarded after use. Antibodies were obtained from Genetex (Zika virus Envelope protein antibody GTX133314) and Fisher Scientific (Goat anti-rabbit IgG Secondary antibody PI31460). Peptides spanning the entire ZIKV Envelope protein as consecutive 15-mers with 12-mer overlap were obtained from BEI Resources (NR-50553).

### ZIKV Genes

The prM and E genes of ZIKV strain Brazil-ZKV2015 (accession #KU497555.1 (SEQ ID NO:9), Asian lineage; polyprotein sequence. SEQ ID NO:8) were inserted into the vIND-ZIKV vaccine candidates. The entire coding region of E (504 amino acids, SEQ ID NO:2) was included in the construct with a methionine amino acid at the N-terminus. For constructs containing prM and E, the 18 amino acids preceding prM (the putative signal sequence within the C protein) were encoded immediately upstream of prM (168 amino acids, SEQ ID NO:3), with a methionine amino acid at the N-terminus. TargetP 1.1 software (Technical University of Denmark) was used to predict the localization of the E protein (e.g., secretory pathway). Variants of the natural capsid SP were selected based on improvements in the output of TargetP 1.1 software. A 6X His tag was encoded immediately downstream of E in all constructs.

### Construction of vIND-ZIKV Plasmids

The ZIKV gene(s) were inserted into a plasmid backbone containing the tetR repressor gene under the control of a constitutive VACV promoter and the tetO₂ operator sequence, which was inserted directly downstream of the natural D6R promoter to control expression of the VACV gene D6R (Hagen, C. J., Titong, A., Sarnoski, E. A. & Verardi, P. H. Antibiotic-dependent expression of early transcription factor subunits leads to stringent control of vaccinia virus replication. Virus research 181, 43-52, doi:10.1016zj.virusres.20I3.12.033 (2014); US20130171189A1). To expedite purification of the recombinant viruses, enhanced green fluorescence protein (EGFP) was also included in the construct under the control of a VACV P₁₁ (FI7R) promoter.

### Generation of vIND-ZIKVs

Recombinant VACVs were generated by infecting BS-C-1 cells in 12-well culture plates with a lac-inducible parental virus (viLacR, expressing DsRed fluorescence protein) for 1 h at room temperature (RT). Infected cells were then overlaid with complete DMEM supplemented with 2.5% FBS containing 0.1 mM isopropyl β-D-1-thiogalactopyranoside (IPTG) and 1 µg/ml DOX. Plasmids were complexed with FuGENE HD transfection reagent (Promega, Madison, WI) for 15 min before being added to individual wells of infected cells. Cells were incubated for 2 days at 37° C., 5% CO₂ before being analyzed with an EVOS FL inverted fluorescence microscope (ThermoFisher Scientific, Waltham, MA) for successful transfection (EGFP expression) and parental virus replication (DsRed expression and cytopathic effect). Cell lysates and supernatants were collected and processed, and vIND-ZIKVs were serially purified from parental virus in the absence of IPTG and presence of DOX by our recently developed method based on the swapping of inducible systems. High-titer stocks were generated by infecting HeLa S3 cells with the VACVs at an MOI of 0.1 in the presence of 1 µg/mL DOX14. The vIND-ZIKVs from high-titer stocks were authenticated by extraction of viral DNA (NucleoSpin Blood Mini kit, Macherey-Nagel, Bethlehem, PA, USA) and PCR amplification with Q5 high-fidelity DNA polymerase (New England Biolabs, Ipswich, MA, USA). The PCR product was checked by restriction enzyme analyses, and either sequenced directly or after cloning into the Zero Blunt PCR cloning kit (Thermo Fisher Scientific). PCR and sequencing primers are shown in Table 1.

TABLE 1

Primer sequences used to authenticate recombinant vaccinia viruses

| Primer | Sequence (5′-3′) | SEQ ID NO. |
|---|---|---|
| PCR Forward 1 | TACTCGAGATGGGCGCAAAG | 10 |
| PCR Forward 2 | ACCTAGCTTCTGGGCGAGTT | 11 |
| PCR Forward 3 | GCCCAACACAAGGTGAAGC | 12 |
| PCR Reverse 1 | CCAGTGCTTCTTTGTTGTTCC | 13 |
| PCR Reverse 2 | TTGTGATGGCAGGTTCCGTA | 14 |
| PCR Reverse 3 | CGCGGTTAGTGATGGTGATG | 15 |
| Sequencing Forward 1 | GTAAAACGACGGCCAG | 16 |
| Sequencing Forward 2 | CCCAAGTTGATGTCGTGTTG | 17 |
| Sequencing Forward 3 | TGACCAAGTATATGACTTTTTGGC | 18 |
| Sequencing Forward 4 | GCAGCTCTAATGCGCTGTTA | 19 |
| Sequencing Reverse 1 | CAGGAAACAGCTATGAC | 20 |
| Sequencing Reverse 2 | AACTTAGATTGAAGGGCGTGTC | 21 |
| Sequencing Reverse 3 | CCACCATTTGGGGACTCTTA | 22 |
| Sequencing Reverse 4 | CCATGATCTGTATATAACAC | 23 |

### Expression of ZIKV Proteins From vIND-ZIKVs by Western Blot

Vero or HeLa S3 cells grown in 100 mm culture dishes to near confluency were infected with each VACV at an MOI of 5. After 1 h, cells were washed and overlaid with D-MEM containing 2.5% FBS with or without the addition of 1 µg/ml DOX, and incubated at 37° C. for 2 days. Cell lysates were collected and processed. Supernatants were clarified by centrifugation (1000 × g for 10 min at 4° C.) and transferred (~8 ml) to conical tubes containing 2 mL of ice-cold 40% PEG-8000, and incubated overnight at 4° C. The 10 ml mixtures were then added to ultraclear centrifuge tubes, loaded onto a SW 32 Ti rotor (Beckman Coulter, Indianapolis, Indiana), and centrifuged at 9,100 rpm for 30 min at 4° C. Supernatants were discarded and pellets were resuspended in 80 µl 10 mM Tris (pH 8.0) buffer.

Samples were run in 4-20% Mini-PROTEAN TGX Stain-Free gels (BioRad, Hercules, California) and proteins were then transferred onto mini PVDF membranes using TransBlot Turbo (BioRad). Membranes were incubated in blocking buffer (5% non-fat milk in PBS-Tween) for 1 h, washed with PBS-Tween, and primary antibody was then added and incubated for 2 h. The membranes were then washed 3 times with PBS-Tween before adding secondary antibody and incubating for 1 hr. Membranes were washed three times with PBS-Tween, two times with water, prepared for chemiluminescent development by incubation in Clarity Western ECL Substrate (BioRad), and imaged with a ChemiDoc digital imager (BioRad).

The effect of DOX on vIND-ZIKV (D4W) plaque formation was determined by infecting near-confluent BS-C-1 cell monolayers in six-well plates with vIND-ZIKV (D4W) at 50 PFU/well in the absence or presence of 1 µg/ml DOX. Individual plaques and infected cells were imaged 2 days later by brightfield and fluorescence microscopy with an Axio Observer D1 inverted fluorescence microscope (Carl Zeiss, Oberkochen, Germany) using an XF100-2 (EGFP) filter (Omega Optical, Brattleboro, VT, USA).

### Negative Staining and Electron Microscopy

In preparation for electron microscopy, concentrated supernatants (described above) were fixed in 2% glutaraldehyde for 15 min. Fixed samples (3 µl) were then added onto plasma-cleaned carbon-coated copper grids (Electron Microscopy Sciences, Hatfield, Pennsylvania) and incubated for 2 min. The grids were then washed with 0.5% uranyl acetate and air dried. Grids were then imaged with a FEI Tecnai 12 G2 Spirit BioTWIN Transmission Electron Microscope at the University of Connecticut Biosciences Electron Microscopy Laboratory.

### One-Step Growth Curve of vIND-ZIKV

BS-C-1 cells were seeded in 12-well culture plates in complete DMEM supplemented with 10% FBS. Cells were washed once with PBS before vIND or vIND-ZIKV (D4W SP) were added to the cells at an MOI of 5 in triplicate for 1 h at RT. After 1 h, cells were washed once with PBS before being overlaid with complete DMEM supplemented with 2.5% FBS and 1 µg/ml DOX. Plates were incubated at 37° C., 5% CO₂. At indicated time points (0 h or 24 h), cell lysates were collected and processed. Processed cell lysates were then diluted and added to fresh BS-C- cells in 24-well culture plates in duplicate to determine viral titer. Infected plates were stained two days later with 0.5% crystal violet/10% ethanol/20% formaldehyde and plaques were enumerated.

### Safety of vIND-ZIKV in Normal Mice

Female CB6F₁ mice (5-week-old) were obtained from Jackson Laboratories. At 6 weeks of age, mice (n=5) were inoculated intranasally with 2 × 10⁴ PFU vIND or vIND-ZIKV (D4W SP) and weighed daily for 21 days. Mice were given either normal drinking water (NO DOX treatment) or 0.125 mg/mL DOX (Sigma-Aldrich, cat. # D9891, ≥98% TLC) in the drinking water, replaced every two days (DOX treatment).

### Immunogenicity and Efficacy of vIND-ZIKV in Mice

To assess CMI responses, 6-week-old C57BL/6 mice (obtained from Jackson Laboratories) were inoculated (n=5) with 10⁷ PFU of vIND, vIND-ZIKV (D4W SP), or PBS intramuscularly in the right hind limb. Mice were sacrificed after seven days and spleens were harvested for ELISPOT analysis. To assess humoral immune responses, 6-week-old C57BL/6 mice (n=8) were vaccinated with 10⁷ PFU of vIND or vIND-ZIKV (D4W SP) and sacrificed two weeks after vaccination. Blood was collected retro-orbitally on day 0 (naive sera) or at euthanasia by cardiac puncture for PRNT. To assess humoral immune responses and efficacy, 6-week-old C57BL/6 mice (n=8) were inoculated with 10⁷ PFU of vIND, vIND-ZIKV (D4W SP), or PBS intramuscularly in the right hind limb. Two weeks later, mice were boosted intramuscularly with 10⁷ PFU of vIND, vIND-ZIKV (D4W SP), or PBS. Two weeks later, mice were challenged with 10⁴ PFU of ZIKV (strain PRVABC59) intraperitoneally. One day prior to challenge, mice were given 2 mg of anti-IFNAR 1 antibody (Leinco Technologies, MAR1-5A3, I-401) intraperitoneally. Mice were bled retro-orbitally on days 0, 14, 27, and 30, and bled via cardiac puncture at euthanasia on day 42. To test the effect of prior vector immunity on the immunogenicity of vIND-ZIKV, 6-week-old C57BL/6 mice (n=8) were first primed intramuscularly with 10⁷ PFU vIND or PBS. Two weeks later, mice were vaccinated to begin the humoral immune responses and efficacy experiment, exactly as described above.

### ELISPOT

First, 96-well ELISPOT plates (BD ELISPOT Mouse IFN-γ ELISPOT Set (BD, San Diego, CA)) were coated with purified anti-mouse IFN-γ overnight at 4° C. Plates were then blocked for at least 2 h at RT with RPMI 1640 containing 10% FBS and 1% Pen-Strep. Next, 4 µg/ml peptide (BEI NR-50553, IGVSNRDFVEGMSGG), which during pilot studies was determined to be the most immunogenic among five peptides tested that contained previously-described H-2^{b} E epitopes (Elong Ngono, A. et al. Mapping and Role of the CD8(+) T Cell Response During Primary Zika Virus Infection in Mice. Cell host & microbe 21, 35-46, doi: 10.1016/j.chom.2016.12.010 (2017); Pardy, R. D. et al. Analysis of the T Cell Response to Zika Virus and Identification of a Novel CD8+ T Cell Epitope in Immunocompetent Mice. PLoS pathogens 13, e1006184, doi:10.1371/journal.ppat.1006184 (2017)), or 5 ng/ml phorbol myristate acetate (PMA) containing 500 ng/ml ionomycin was added to the well followed by 2 × 10⁵ freshly harvested splenocytes. The cells were incubated for 18 h at 37° C., 5% CO₂. Cell suspensions were aspirated and plates were washed twice with water and three times with PBS-Tween before adding biotinylated anti-mouse IFN-y. After incubation for 2 hours at RT, plates were washed 3 times with PBS-Tween before Streptavidin-HRP was added. After 1 h incubation at RT. wells were washed four times with PBS-Tween, twice with PBS, and BD AEC Substrate kit was added. Spot development was monitored and stopped after 15 min by washing wells with water. Plates were air-dried overnight before spots were counted manually after imaging with a stereoscope.

### Plaque Reduction Neutralization Assay (PRNT)

To measure the ability of serum to neutralize ZIKV, PRNTs were performed. Briefly, 12-well cell culture plates were seeded with Vero cells so that they were near confluency at the time of infection. Serum was heat-inactivated at 37° C. for 30 min. Serum samples collected during the immunogenicity study (at euthanasia) were analyzed individually, while serum collected during the efficacy studies were pooled due to low volumes (periodic retro-orbital bleeding). Serum was diluted 2-fold in complete DME containing 1X antibiotic-antimycotic (Life Technologies) and mixed with equal volumes of ZIKV strain PRVABC59 containing approximately 50 PFU/well. Serum/virus dilutions were incubated for 1 h at 37° C., 5% CO₂. After incubation, cells were infected with the serum/virus dilutions in duplicates for 1 h, inoculum was then aspirated and cells were overlaid with complete DME containing 1X antibiotic-antimycotic, 2.5% FBS, and 1% methylcellulose. Plates were incubated for 4 days at 37° C. with 5% CO₂ prior to fixation with 0.5% crystal violet in 10% ethanol/20% formaldehyde and manual plaque counting. The PRNT₅₀ was calculated as the reciprocal of the dilution that resulted in at least 50% reduction in ZIKV plaques.

### ELISAs

To detect antibodies against ZIKV, serum collected from mice was diluted 1:100, 1:500, 1:1000, 1:4000, or 1:5000 for ELISA. Recombivirus Mouse Anti-Zika Virus Envelope Protein IgG kit (Alpha Diagnostic International, RV-403120-1) and Recombivirus Mouse Anti-Zika Virus NS1 Protein IgG kit (Alpha Diagnostic International, RV-403320-1) were performed according to manufacturer’s instructions. Optical density (OD) was measured at 450 nm with a reference wavelength of 630 nm. Antibody concentrations (U/ml) were calculated based on a standard curve. Lower limit of detection (LLD) was 100 U/ml. To detect antibodies against VACV, an in-house ELISA was developed. Briefly, flat-bottom 96-well plates were coated with VACV strain WR (~ 2 × 10⁴ PFU) diluted in 100 µl PBS containing 0.1 % FBS and incubated overnight at 4° C. Serum collected from mice was pooled due to low volumes and subsequently serially diluted twofold in PBS containing 5% non-fat milk and 0.05% Tween. Plates were washed and blocked for 1 h in PBS containing 5% non-fat milk and 0.05% Tween. Plates were washed, serial dilutions of serum were added, and plates were incubated for 2 h. Plates were then washed, anti-mouse IgG-HRP conjugate (31430, Invitrogen, Carlsbad, CA, USA) diluted 1:1000 in PBS containing 5% non-fat milk and 0.05% Tween-20 was added, and plates were incubated for 1 h. Plates were washed, TMB Substrate (N301, Thermo Fisher Scientific) was added, the reaction was stopped with 2 M H₂SO₄, and OD was measured at 450 nm. Endpoint titers were calculated as the reciprocal of the highest serum dilution that gave a reading above the cutoff (upper prediction limit of a Student t-distribution of the no-serum control readings at 95% confidence interval).

### Analysis of ZIKV Viremia by qRT-PCR

Blood was collected by retro-orbital bleeding to analyze ZIKV viremia two days after challenge. RNA was extracted from 20 µl mouse serum using the QIAamp Viral Mini Kit (Qiagen, Venlo, Netherlands) per the manufacturer’s instructions. qRT-PCR was performed on the RNA in triplicate using iTaq Universal Probes One-Step Kit (BioRad, Hercules, California) with primers previously described (Lanciotti, R. S. et al. Genetic and serologic properties of Zika virus associated with an epidemic, Yap State, Micronesia, 2007. Emerging infectious diseases 14, 1232-39, doi:10.3201/eid1408.080287 (2008)). PFU equivalents were calculated using a standard curve prepared from a previously titrated sample of the ZIKV strain PRVABC59.

### Statistical Analyses

Statistical analyses were performed using GraphPad Prism v.7.0e software (GraphPad Software, La Jolla, CA). A p value less than 0.05 was considered statistically significant.

### Results

### Design and Generation of ZIKV Vaccine Candidates

Several vaccine candidates were generated against ZIKV based on a sequenced isolate (Asian lineage), Brazil-ZKV2015 (accession #KU497555.1) (Calvet, G. et al. Detection and sequencing of Zika virus from amniotic fluid of fetuses with microcephaly in Brazil: a case study. The Lancet. Infectious diseases 16, 653-60, doi: 10.1016/S1473-3099(16)00095-5 (2016)). A schematic representation of the vaccine constructs is shown in FIG. 1a. The ZIKV gene(s) were placed under the control of a synthetic VACV P_{E/L} promoter (Chakrabarti, S., Sisler. J. R. & Moss, B. Compact, synthetic, vaccinia virus early/late promoter for protein expression. Biotechniques 23, 1094-97, doi:10.2144/97236st07 (1997)) and inserted between VACV genes D5R and D6R by homologous recombination, generating a vIND that replicates only in the presence of tetracyclines (Hagen et al., 2014). Enhanced green fluorescence protein (EGFP) was included in the recombinant VACVs (rVACVs) to expedite purification. The first vaccine candidate contained the full-length Envelope (E) protein with a methionine added immediately upstream to facilitate translation. A second vaccine candidate was designed that included prM and E, along with the putative natural signal peptide (SP) encoded in the last 18 amino acids of C (Kuno & Chang, 2007), to ensure proper folding and secretion of E and lead to the formation of VLPs (Mukhopadhyay, Kuhn, & Rossmann, 2005) (FIG. 1a).

SPs characteristically contain three distinct domains: an N-terminal (n) region often containing positively-charged residues, a hydrophobic (h) region of at least six hydrophobic residues, and a polar uncharged C-terminal (c) region (Emanuelsson, O., Brunak, S., von Heijne, G. & Nielsen, H. Locating proteins in the cell using TargetP, SignalP and related tools. Nature protocols 2, 953-71, doi:10.1038/nprot.2007.131 (2007)) to facilitate translocation into the endoplasmic reticulum (ER) and in the case of ZIKV capsid SP, to direct prM into the ER lumen for proper secretion of E. Upon reviewing the ZIKV SP sequence, the negatively-charged aspartic acid (D) in the n-region (FIG. 1a) would lead to sub-optimal secretion of E caused concern. A series of SP variants was designed using TargetP 1.1 software (Emanuelsson et al., 2007) to evaluate the localization of proteins based on the SP sequence. The first variant generated replaced the aspartic acid residue with a strongly hydrophobic residue, of which, tryptophan (W) resulted in the highest secretory pathway prediction score (0.930), compared to the natural SP (score 0.865). Also generated was a SP variant that replaced the aspartic acid with a positively charged lysine (K) (score 0.878). Lastly, a vaccine candidate was generated that included the last 22 amino acids of the SP of Japanese Encephalitis Virus (JEV, score 0.931), since this sequence has been used successfully to target proteins for secretion (Chang, G. J., Hunt, A. R. & Davis, B. A single intramuscular injection of recombinant plasmid DNA induces protective immunity and prevents Japanese encephalitis in mice. Journal of virology 74, 4244-52 (2000)).

Once constructs containing the desired ZIKV antigens were generated (FIG. 1a), they were subcloned into a plasmid containing elements of the tetracycline (tet) operon (Hagen et al., 2014) to facilitate generation of vINDs expressing the ZIKV antigens (vIND-ZIKVs). The resulting shuttle vectors were transfected into cells infected with a lac-inducible parental virus, and after homologous recombination, vIND-ZIKVs were purified away from parental virus using our recently developed accelerated method. Briefly, cells were serially infected with the parental VACV/rVACV pool in the presence of DOX (rVACV inducer) and absence of isopropyl β-D-1-thiogalactopyranoside (IPTG, parental VACV inducer). Using this method, single clones of each vIND-ZIKV were obtained. Nucleic acid sequences of each of the five vIND-ZIKV constructs depicted schematically in FIG. 1a are included as SEQ ID Nos:32-36, respectively. Positioning of the various elements depicted in FIG. 1a in the sequences are summarized in Tables 2-4 below.

TABLE 2

Sequence positions of elements of vIND-ZIKV construct including only ZIKV E protein (SEQ ID NO:32)

| Element | Range |
|---|---|
| D5R | 1 - 600 |
| EGFP | 614 - 1333 |
| P₁₁ | 1340 - 1381 |
| tetR | 1395 - 2018 |
| P_{E/L} | 2025 - 2066 |
| P_{E/L} | 2073 - 2114 |
| ZIKV E alone | 2121 - 3635 |
| 6X His tag | 3636 - 3656 |
| P_{D6R} | 3704 - 3746 |
| tetO₂ | 3747 - 3765 |
| D6R | 3772 - 4371 |

TABLE 3

Sequence positions of elements of vIND-ZIKV constructs including the natural (wild type) ZIKV SP (SEQ ID NO:33), D4W SP (SEQ ID NO:34), or D4K SP (SEQ ID NO:35)

| Element | Range |
|---|---|
| D5R | 1 - 600 |
| EGFP | 614 - 1333 |
| P₁₁ | 1340 - 1381 |
| tetR | 1395 - 2018 |
| P_{E/L} | 2025 - 2066 |
| P_{E/L} | 2073 - 2114 |
| ZIKV signal sequence (natural, D4W, D4K) | 2121 - 2177 |
| ZIKV prM | 2178 - 2681 |
| ZIKV E | 2682 - 4193 |
| 6X His tag | 4194 - 4214 |
| P_{D6R} | 4262 - 4304 |
| tetO₂ | 4305 - 4323 |
| D6R | 4330 - 4929 |

TABLE 4

Sequence positions of elements of vIND-ZIKV constructs including only the JEV SP (SEQ ID NO:36)

| Element | Range |
|---|---|
| D5R | 1 - 600 |
| EGFP | 614 - 1333 |
| P₁₁ | 1340 - 1381 |
| tetR | 1395 - 2018 |
| P_{E/L} | 2025 - 2066 |
| P_{E/L} | 2073 - 2114 |
| ZIKV signal sequence (JEV) | 2121 - 2189 |
| ZIKV prM | 2190 - 2693 |
| ZIKV E | 2694 - 4205 |
| 6X His tag | 4206 - 4226 |
| P_{D6R} | 4274 - 4316 |
| tetO₂ | 4317 - 4335 |
| D6R | 4342 - 4941 |

### Single Mutations Within the SP of prM Result in Increased Secretion of E

The five vaccine candidates (FIG. 1a) were then evaluated for expression of E in both the lysate and supernatant of infected Vero cells by western blot (FIG. 1b). A protein matching the expected size of E (-55 kDa) was observed in the cell lysate for all vaccine constructs, albeit to different levels. In cells infected with the vIND- ZIKV expressing E only (without SP and prM) expression of E was contained within the cell (cell lysate), with little secretion of E detected in the supernatant (FIG. 1b). Similarly, in cells infected with the vIND- ZIKV expressing the natural SP, prM, and E. low levels of E were secreted into the supernatant by the natural SP, but those levels increased dramatically when the natural SP was replaced with each of the three variant signal peptides, with the D4W SP showing the greatest enhancement.

### vIND-ZIKVs Produce ZIKV VLPs

vIND-ZIKV was also evaluated for formation of VLPs by transmission electron microscopy (TEM) compared to wild-type ZIKV particles. Stock ZIKV strain PRY ABC59 or supernatant from cells infected with vIND-ZIKVs were concentrated and fixed with 2% glutaraldehyde, loaded onto grids, and negatively stained with 0.5% uranyl acetate for TEM imaging. VLPs of the expected size (-50-60 nm (Hasan, S. S., Sevvana, M., Kuhn, R. J. & Rossmann, M. G. Structural biology of Zika virus and other flaviviruses. Nat Struct Mol Biol 25. 13-20, doi: 10.1038/s4l594-017-0010-8 (2018))) we visualized in the supernatant of vIND-ZIKV-infected cells that resembled virions produced by ZIKV PRVABC59 (D4W SP mutant shown in FIG. 1d).

### vIND-ZIKV Grows to High Titers in the Presence of DOX but Does not Replicate in the Absence of DOX

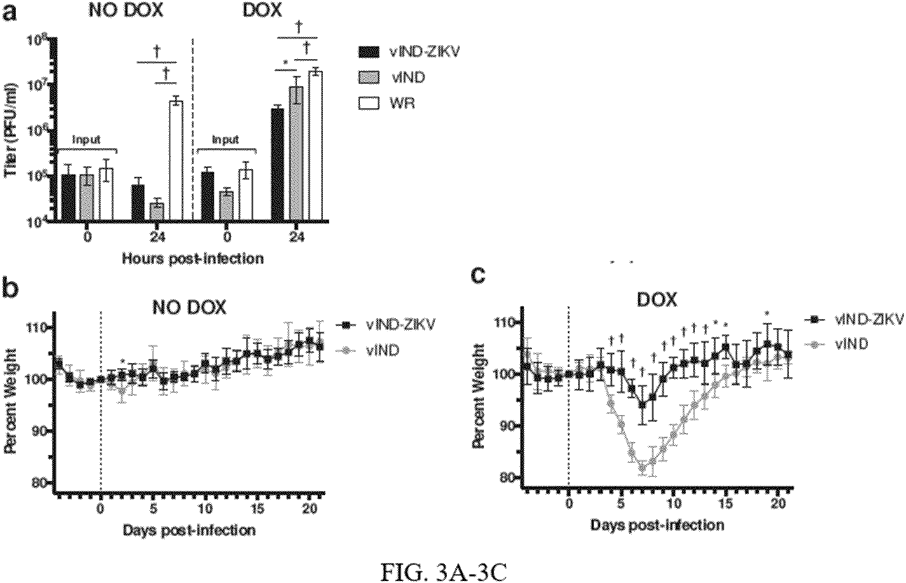

Because the vIND-ZIKV containing the D4W SP variant resulted in dramatically increased expression of E in both the cell lysate and supernatant, this vINO-ZIKV was selected as the vaccine candidate to progress to further studies (referred from now on as vIND-ZIKV). Next, the replication of vIND-ZIKV was evaluated in vitro (FIG. 3a). BS-C-1 cells were infected with vIND-ZIKV, vIND, or the wild-type (replication-competent) strain Western Reserve (WR) at a multiplicity of infection (MOI) of 5 in the absence or presence of 1 µg/ml doxycycline (DOX). Cells were collected at 0 and 24 h post infection (hpi) and lysates were titered on fresh monolayers in the presence of DOX. In the absence of DOX, vIND-ZIKV and vIND did not replicate (titers at 24 hpi were lower than input levels at 0 hpi), while the replication-competent WR replicated to high titers. In the presence of DOX, _{V}IND reached near-wildtype levels of replication by 24 hpi, albeit statistically significantly lower than WR; however, vIND-ZIKV replication was attenuated compared to both WR and vIND (p<0.001 and p<0.05, respectively). Despite the attenuation of vIND-ZIKV in vitro, high titer vaccine stocks were still readily generated in the presence of DOX for downstream studies.

### vIND-ZIKV Is Attenuated in Mice Even in the Presence of DOX

To evaluate the safety of vIND-ZIKV. 6-week-old CB6F₁ mice were inoculated intranasally with 2 × 10⁴ PFU vIND-ZIKV or vIND in either the absence or presence of DOX in the drinking water and were weighed daily for 21 days (FIGS. 3b and c, respectively). Intranasal infection of normal mice is an ideal route for studies of poxvirus pathogenesis and virulence, since replication-competent VACVs lead to infection of the central nervous system and weight loss (Williamson, J. D., Reith, R. W., Jeffrey, L. J., Arrand, J. R. & Mackett, M. Biological characterization of recombinant vaccinia viruses in mice infected by the respiratory route. The Journal of general virology 71 (Pt 11). 2761-67, doi: 10.1099/0022-1317-71-11-2761 (1990), and this dose was shown to cause weight loss without mortality in vIND-infected mice during pilot studies. vINDs are replication-defective in the absence of DOX and should therefore be safer, yet they cause weight loss and mortality (with intranasal inoculation) and replicate to wild-type levels in ovaries (with intraperitoneal inoculation) in the presence of DOX (as do replication-competent VACVs).. Accordingly, in the absence of DOX, mice in both groups maintained and then gained weight throughout the study (FIG. 3b). In the presence of DOX, vIND-infected mice started to lose weight on day 4, reached peak weight loss at day 7, and recovered back to starting weight by day 16 (FIG. 3c). However, vIND-ZIKV was slightly more attenuated than vIND in the presence of DOX, as mice infected with vIND-ZIK V lost weight to a lesser degree than those infected with vIND (p<0.001). This demonstrated that our vIND-ZIKV vaccine would be safe when given as a vaccine in the absence of DOX and has an added safety feature, since it is attenuated (compared to vIND) even in the presence of DOX.

### vIND-ZIKV Induces High Levels of Cell-Mediated Immune Responses in Mice

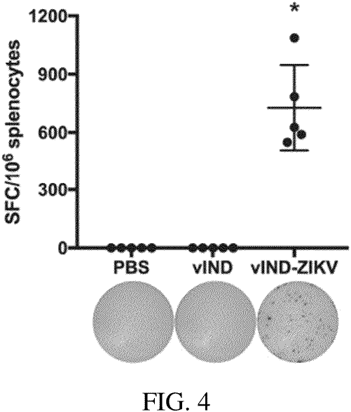

An evaluation of the immunogenicity of the vaccine candidate was then conducted. Cell-mediated immunity (CMI) was tested by vaccinating 6-week-old C57BL/6 mice (n=5) intramuscularly with 10⁷ PFU vIND, vIND-ZIKV, or PBS (in the absence of DOX). After seven days, mice were sacrificed and spleens were removed and splenocytes were harvested. Freshly isolated splenocytes were incubated with 4 µg/ml of a 15-mer peptide of ZIKV E protein for 18 h for an ELISPOT assay. Mice vaccinated with vIND-ZIKV had robust levels of antigen-specific IFN--γ-secreting splenocytes that were not detected in mice vaccinated with PBS (p<0.01) or vIND (FIG. 4).

### Humoral Immune Responses of vIND-ZIKV in Mice

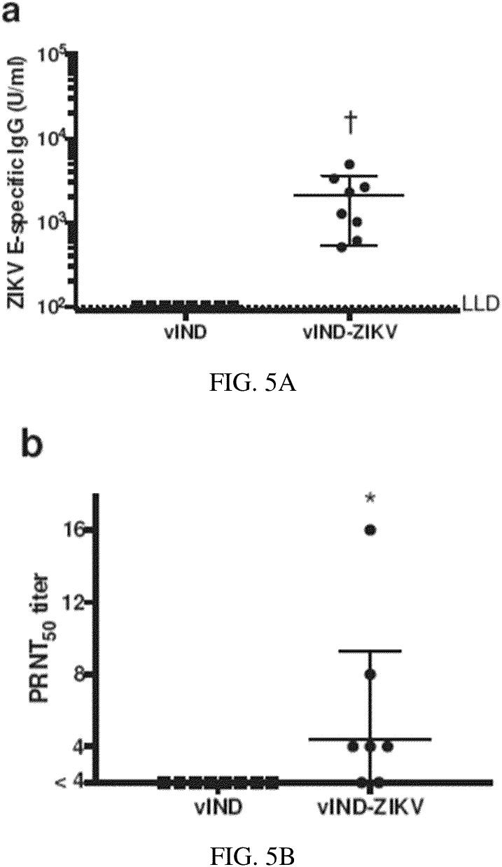

The humoral immune responses of vIND-ZIKV were analyzed by measuring the induction of ZIKV E-specific IgG and neutralizing antibodies (FIG. 5). Six-week-old C57BL/6 mice (n=8) were vaccinated intramuscularly with 10⁷ PFU vIND or vIND-ZIKV. Blood was collected on the day of vaccination (naive sera) or at euthanasia (4 weeks after vaccination) for analysis. Antibodies against ZIKV E were measured by ELISA (FIG. 5a). vIND-vaccinated mice had no E-specific IgG titers after vaccination, while vIND-ZIKV vaccination induced robust levels of E-specific antibodies (geometric mean 2,072 U/ml, p<0.001). Next, neutralizing antibodies in serum were measured by plaque reduction neutralization test (PRNT) (FIG. 5b). As expected, serum from vIND-vaccinated mice did not neutralize ZIKV (PRNT₅₀ titer < 4). Surprisingly, mice inoculated with vIND-ZIKV had low neutralizing antibody titers (geometric mean PRNT₅₀ titer of 4.4) after vaccination, although they were statistically higher than vIND (p<0.01). One vIND-ZIKV-vaccinated mouse was excluded from analysis due to low volume of serum collected that prevented analysis at the lowest dilution, although this mouse had a PRNT₅₀ titer < 6. Despite low neutralizing antibody titers, vIND-ZIKV vaccination induced robust levels of E-specific IgG (FIG. 5a) and antigen-specific CMI (FIG. 4) that warranted further investigation in a challenge model.

### vIND-ZIKV Induces Humoral Immune Responses and Protects Mice From Viremia

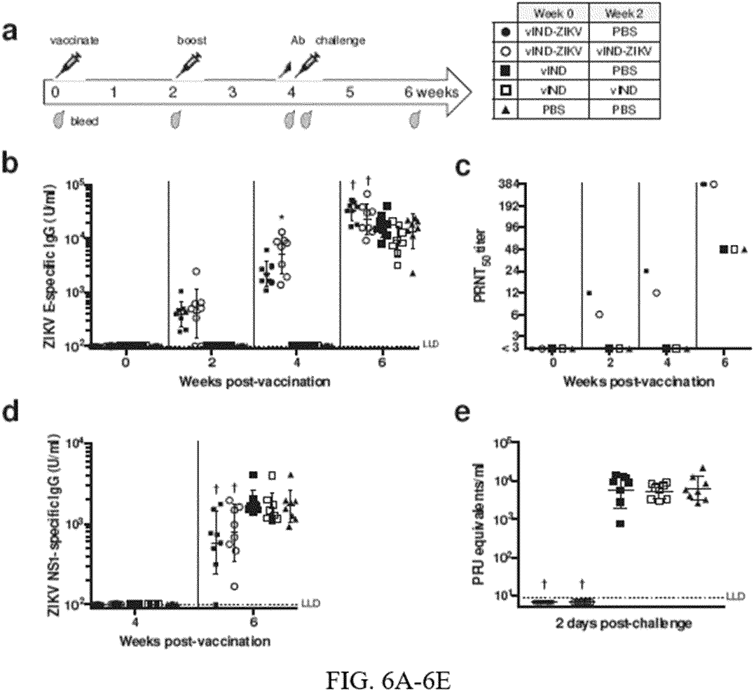

To further assess the humoral immune responses and simultaneously evaluate efficacy of viND-ZIKV, a recently-developed ZIKV challenge model (Lazear, H. M. et al. A Mouse Model of Zika Virus Pathogenesis. Cell host & microbe 19, 720-30, doi:10.1016/j.chom.2016.03.010 (2016)) was utilized, wherein C57BL/6 mice are made transiently susceptible to ZIKV infection by administering an IFNAR1 monoclonal antibody (Sheehan, K. C. et al. Blocking monoclonal antibodies specific for mouse IFN-alpha/beta receptor subunit 1 (IFNAR-1) from mice immunized by in vivo hydrodynamic transfection. Journal of interferon & cytokine research: the official journal of the International Society for Interferon and Cytokine Research 26, 804-19, doi:10.1089/jir.2006.26.804 (2006)) one day prior to challenge. Since low neutralizing antibody titers were observed after a single vIND. ZIKV vaccination (FIG. 5b), a group receiving two vIND-ZIKV vaccinations was included (as indicated in FIGS. 6). Briefly, 6-week-old C57BL/6 mice (n=8) were vaccinated intramuscularly with PBS or 10⁷ PFU vIND or vIND-ZIKV at weeks 0 and 2, as outlined in FIG. 6a. Mice were challenged 2 weeks post-boost with 10⁴ PFU ZIKV strain PRVABC59 (Asian lineage) intraperitoneally, 1 day after administration of 2 mg anti-IFNAR 1 antibody (Leinco, MAR1-5A3) intraperitoneally. Mice were sacrificed 2 weeks later at the conclusion of the study. Blood was collected retro-orbitally at regular intervals (FIG. 6a) or by cardiac puncture at euthanasia.

To measure the humoral immune response to vIND-ZIKV. antibody titers against E were analyzed by ELISA (FIG. 6b). In mice vaccinated once with vIND-ZIKV, E-specific IgG titers were low (geometric mean 387 U/ml) 2 weeks post vaccination, but increased by 4 weeks post vaccination (geometric mean 2,166 U/ml). Similarly, mice vaccinated twice with vIND-ZIKV had low anti-E titers following the first vaccination (geometric mean 411 U/ml), but increased 10-fold after a second vaccination with vIND-ZIKV (geometric mean 5,214 U/ml; p<0.05 compared to mice inoculated with PBS). Following ZIKV challenge, control groups vaccinated with vIND (once or twice) or PBS developed anti-E IgG titers (geometric mean of 17,291; 9,078; or 13,578 U/ml, respectively). E-specific antibody titers were boosted post-challenge in mice vaccinated once or twice with vIND-ZIKV (geometric mean 32,509 or 22,964 U/ml, respectively), and were statistically significantly higher than mice inoculated with PBS (geometric mean 13,578 U/ml; p<0.005) (FIG. 6b).

The level of neutralizing antibodies was determined for each group at weeks 0, 2, 4, and 6. Since only small volumes of blood were collected retro-orbitally at each time point, serum from each group was pooled for PRNT analysis. Mice vaccinated with vIND-ZIKV had a modest increase in neutralizing titer at weeks 2 and 4 followed by an increase after challenge (FIG. 6c). Next, to assess the extent to which vIND-ZIKV protected against ZIKV replication after challenge, antibody titers against NS1 were measured by ELISA. Most mice vaccinated with vIND-ZIKV once or twice had detectable NS1 titers at week 6 (2 weeks post-challenge), although significantly lower when compared to PBS-vaccinated controls (p<0.005) (FIG. 6d), indicating potential challenge virus replication (i.e., lack of sterilizing immunity).

C57BL/6 mice made transiently susceptible to ZIKV by anti-IFNAR1 antibody develop viremia after challenge (Lazear et al., 2016). Blood was collected two days post-challenge and performed quantitative reverse transcription PCR (qRT-PCR) using primers previously described (Lanciotti et al., 2008). Mice vaccinated with PBS or vIND once or twice had high levels of viremia (FIG. 6e). However, mice vaccinated once or twice with vIND-ZIKV were protected from viremia (p<0.005). Therefore, a single dose of vIND-ZIKV was sufficient to completely protect mice from transient viremia.

### VACV-Primed Mice Vaccinated Twice With vIND-ZIKV Are Protected From Viremia

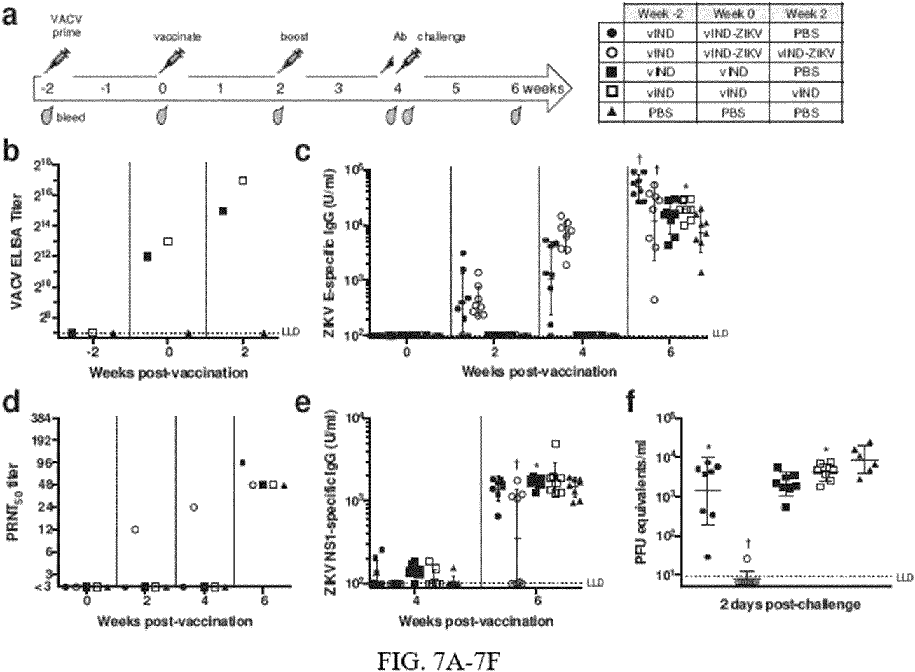

Since VACV can be, and has been, used for many applications (e.g., vaccine, therapeutic, and oncolytic vectors), the present applicants also wanted to explore if prior immunity to VACV had any impact on the immunogenicity and efficacy of our ZIKV vaccine. To test this, the vaccination/challenge experiment described above was repeated but added a “VACV prime” 2 weeks prior to vaccination by inoculating mice intramuscularly with 10⁷ vIND (or PBS), in the absence of DOX, to mirror prior vector immunity (FIG. 7a). Anti-VACV antibodies were detected in vIND-primed mice at the time of vaccination (week 0) and were further increased by week 2 (FIG. 7b).

As seen previously in naive mice (FIG. 6b), mice vaccinated once with vIND-ZIKV had low E-specific IgG titers 2 weeks post vaccination (geometric mean 394 U/ml), but titers increased by 4 weeks post vaccination (mean 1,084 U/ml) (FIG. 7c). Mice vaccinated twice with vIND-ZIKV had similarly low anti-E titers 2 weeks after initial vaccination (geometric mean 413 U/ml), which increased nearly 15-fold after the booster vaccination (geometric mean 6,050 U/ml). Control groups vaccinated once or twice with vIND, or with PBS only developed anti-E IgG titers after ZIKV challenge (geometric mean of 13,456; 19,521; or 7,477 U/ml; respectively). Antibody titers against E increased after ZIKV challenge in mice vaccinated once or twice with vIND-ZIKV (geometric mean 48,884 or 11,719 U/ml, respectively), and were statistically significantly higher than mice vaccinated with PBS (p<0.005) (FIG. 7c). As seen in the previous challenge study, mice vaccinated with PBS or vIND developed neutralizing antibody titers only after challenge (FIG. 7d). Interestingly, only mice vaccinated twice with vIND-ZIKV had detectable neutralizing antibody titers in pooled sera at weeks 2 and 4, which increased after challenge (FIG. 7d). Similarly, mice vaccinated twice with vIND-ZIKV had reduced NS1-specific antibody titers 2 weeks post-challenge (FIG. 7d).

As above, blood was collected two days post-challenge and performed qRT-PCR to measure ZIKV viremia. As expected, VACV-primed mice vaccinated with PBS or vIND had high levels of viremia (FIG. 7f). Interestingly, VACV-primed mice vaccinated once with vIND-ZIKV had high levels of viremia, showing that prior inoculation with vIND in the absence of DOX most likely resulted in vector immunity that interfered with vIND-ZIKV vaccination. This finding corresponded with the lack of detectable ZIKV neutralizing antibody titer in this group (FIG. 7d). However, a second vaccination with vIND-ZIKV protected VACV-primed mice from viremia, as levels were statistically significantly lower than the PBS control group (p<0.005), and were close to or below the detection limit (FIG. 7f).

### Example 2. POWV Vaccines

Several vaccine candidates were generated against POWV by methods analogous to those described in Example 1. The POWV sequences were based on a sequenced isolate. POWV strain LB. The polyprotein for the POWV LB strain is UNIPROT Q04538,having the sequence of SEQ ID NO:28. The sequence of the gene of the POWV LB strain polyprotein is GENBANK MF374486.1, SEQ ID NO: 31. The amino acid sequences of the prM protein and E protein of the POWV LB strain are SEQ ID No:30 and SEQ ID NO:29, respectively.

FIG. 2a is a schematic representation of vIND-POWV vaccine constructs in the D5R-D6R locus of VACV. Constructs contained the tet repressor gene (tetR) under the control of a strong synthetic early/late promoter (P_{E/L}) and the tet operator sequence (O₂) immediately downstream of the natural D6R promoter (P_{D6R}). The POWV prM and E gene(s) were placed under the control of a synthetic VACV P_{E/L} promoter and inserted between VACV genes D5R and D6R by homologous recombination, generating a vIND that replicates only in the presence of tetracyclines. The dsRed gene was included in these recombinant VACVs (rVACVs) to expedite purification. Four POWV vaccine candidates with the natural POWV SP fMRSGVDWTWIFLTMALTMAMAT, SEQ ID NO:24), the POWV D6W mutant SP (MRSGVWWTWIFLTMALTMAMAT, SEQ ID NO:25), the POWV D6K mutant SP (MRSGVKWTWIFLTMALTMAMAT, SEQ ID NO:26), or the JEV SP (MGGNEGSIMWLASLAVVIACAGA, SEQ ID NO:4) were generated and tested for secretion of E protein and formation of VLPs. Nucleic acid sequences of each of the four vIND-POWV constructs depicted schematically in FIG. 2a are included as SEQ ID Nos:37-40, respectively. Positioning of the various elements depicted in FIG. 2a in the sequences are summarized in Table 5 below.

TABLE 5

Sequence positions of elements of vIND-POWV constructs including the natural (wild type) POWV SP (SEQ ID NO:37), D6W SP (SEQ ID NO:38), or D6K SP (SEQ ID NO:39), or JEV SP(SEQ ID NO:40

| Element | Range |
|---|---|
| D5R | 1-600 |
| tetR | 617-1237 |
| P_{E/L} | 1244-1285 |
| P_{E/L} | 1292-1333 |
| POWV signal sequence | 1334-1402 |
| POWV prM | 1403-1891 |
| POWV E | 1892-3385 |
| Psel | 3386-3477 |
| DsRed | 3478-4155 |
| P_{D6R} | 4162-4204 |
| tetO₂ | 4205-4223 |
| D6R | 4230-4829 |

The results are shown in FIGS. 2. FIGS. 2 panel b is a Western blot of supernatants of Vero cells infected with the POWV vaccine candidates including natural POWV SP, JEV SP, or the POWV D6W mutant SP. Bands of approximately 55 kDa were observed. FIGS. 2 panel c presents representative TEM images of POWV VLPs secreted into the supernatant of vIND-POWV (natural SP)-infected Vero cells.

Mice are vaccinated with vIND-POWV (POWV D4W mutant SP), vIND-POWV (POWV D6K mutant SP), or vIND-POWV (JEV SP) as described in Example 1. The vIND-POWV (POWV D4W mutant SP), vIND-POWV (POWV D6K mutant SP), and vIND-POWV (JEV SP) vaccines are each shown to be effective in inducing immune responses against the POWV-specific antigens.

In general, the compositions and methods described here can alternatively comprise, consist of, or consist essentially of, any components or steps herein disclosed. The compositions and methods can additionally, or alternatively, be manufactured or conducted so as to be devoid, or substantially free, of any ingredients, steps, or components not necessary to the achievement of the function or objectives of the present claims.

The singular forms “a,” “an,” and “the” include plural referents unless the context clearly dictates otherwise. “Or” means “and/or.” The values described herein are inclusive of an acceptable error range for the particular value as determined by one of ordinary skill in the art, which will depend in part on how the value is measured or determined, i.e., the limitations of the measurement system. The endpoints of all ranges directed to the same component or property are inclusive of the endpoints and intermediate values, and independently combinable.

Unless defined otherwise, technical and scientific terms used herein have the same meaning as is commonly understood by one of skill in the art to which this disclosure belongs.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While the disclosed subject matter is described herein in terms of some embodiments and representative examples, those skilled in the art will recognize that various modifications and improvements can be made to the disclosed subject matter without departing from the scope thereof. Additional features known in the art likewise can be incorporated. Moreover, although individual features of some embodiments of the disclosed subject matter can be discussed herein and not in other embodiments, it should be apparent that individual features of some embodiments can be combined with one or more features of another embodiment or features from a plurality of embodiments.

1. An engineered signal peptide comprising the amino acid sequence X₁GAX₂TSVGIV GLLLTTAMA (SEQ ID NO:1) or the amino acid sequence X₁RSGVX₂WTWIFLTMALTMAMAT (SEQ ID NO:27), wherein X₁ is M or absent and X₂ is A, I, L, M, F, H, V, P, G, Y, W, R, or K.

2. The signal peptide according to claim 1, wherein X₁ is M and X₂ is W or K.

3. The signal peptide according to claim 1, wherein X₁ is M and X₂ is W.

4. The signal peptide according to claim 1 comprising the amino acid sequence X₁GAX₂TSVGIV GLLLTTAMA (SEQ ID NO:1)or X₁RSGVX₂WTWIFLTMALTMAMAT (SEQ ID NO:27).

5. (canceled)

6. A fusion polypeptide comprising the engineered signal peptide of claim 1 and a flavivirus envelope (E) protein.

7. The fusion polypeptide according to claim 6, further comprising a flavivirus pre-membrane (prM) protein.

8. The fusion polypeptide according to claim 7, wherein the N-terminus to C-terminus ordering is an engineered signal peptide-prM-E.

9. The fusion polypeptide according to claim 6, wherein the flavivirus is selected from one or more of Zika virus, dengue virus, yellow fever virus, Powassan virus, West Nile virus, Japanese encephalitis virus, and tick-borne encephalitis virus.

10. The fusion polypeptide according to claim 6, wherein the E protein has the amino acid sequence of SEQ ID NO:2 or at least 90% identical to SEQ ID NO:2; or wherein the E protein has the amino acid sequence of SEQ ID NO:29 or at least 90% identical to SEQ ID NO:29.

11. (canceled)

12. The fusion polypeptide according to claim 7, wherein prM protein has the amino acid sequence of SEQ ID NO:3 or at least 90% identical to SEQ ID NO:3; or wherein prM protein has the amino acid sequence of SEQ ID NO:30 or at least 90% identical to SEQ ID NO:30.

13. (canceled)

14. A polynucleotide encoding the fusion polypeptide of claim 6.

15. An expression vector comprising a polynucleotide encoding the fusion polypeptide of claim 6.

16. The expression vector of claim 15, which comprises a recombinant replication-inducible vaccinia virus (vIND).

17. The expression vector of claim 16, wherein the vIND comprises tetracycline operon elements and replicates only in the presence of a tetracycline.

18. The expression vector of claim 16, wherein the vIND comprises the sequence of any one of SEQ ID NOs:32-40.

19. A pharmaceutical composition comprising the expression vector of claim 15 and a pharmaceutically acceptable carrier.

20. (canceled)

21. A method of vaccinating a subject against a flavivirus infection, treating a flavivirus infection, or reducing risk of contracting a flavivirus infection comprising administering pharmaceutical composition of claim 19.

22. (canceled)

23. (canceled)

24. (canceled)

25. A method of producing flavivirus virus-like particles (VLPs), wherein the method comprises introducing the expression vector of claim 15 into a cell; culturing the cell under conditions permitting expression of the fusion protein and production of virus-like particles (VLPs); and isolating the VLPs.

26. The method of claim 25, wherein the cell is a mammalian, insect, or yeast cell.

27. The method of claim 21, wherein the flavivirus is selected from one or more of Zika virus, dengue virus, yellow fever virus, Powassan virus, West Nile virus, and tick-borne encephalitis virus.

28. (canceled)

29. (canceled)


SEQUENCE LISTING (40 sequences)

SEQ ID NO: 1 - protein, 19 aa, artificial sequence
  synthetic construct (engineered SP genus sequence)
  MISC_FEATURE (1)..(1): X1=M or absent
  MISC_FEATURE (4)..(4): X4=A, I, L, M, F, H, V, P, G, Y, W, R, or K
XGAXTSVGIVGLLLTTAMA

SEQ ID NO: 2 - protein, 504 aa, Zika Virus Brazil-ZKV2015
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGTQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 3 - protein, 168 aa, Zika Virus Brazil-ZKV2015
AEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYS

SEQ ID NO: 4 - protein, 23 aa, artificial sequence
  synthetic construct (M+JEV sp)
MGGNEGSIMWLASLAVVIACAGA

SEQ ID NO: 5 - protein, 19 aa, artificial sequence
  synthetic construct (M+M+ZIKA virus Brazil-ZKV2015 signal peptide)
MGADTSVGIVGLLLTTAMA

SEQ ID NO: 6 - protein, 19 aa, artificial sequence
  synthetic construct (D4W)
MGAWTSVGIVGLLLTTAMA

SEQ ID NO: 7 - protein, 19 aa, artificial sequence
  synthetic construct (D4K))
MGAKTSVGIVGLLLTTAMA

SEQ ID NO: 8 - protein, 3423 aa, Zika Virus Brazil-ZKV2015
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGTQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCFLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYVAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 9 - DNA, 10793 bp, Zika Virus Brazil-ZKV2015
ccaatctgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgccaggaaggagaagaagagacgaggcgcagatactagtgtcggaatcgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatgggggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcttaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggactcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgtggtacaggggtgttcgtctataacgacgttgaagcctggagggacaggtacaagtaccatcctgactctccccgtagattggcagcagcagtcaagcaagcctgggaagatggtatctgcgggatctcctctgtttcaagaatggaaaacatcatgtggagatcagtagaaggggagcttaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgttttttgcaaactgcgatctccgccttggaaggcgacctgatggttctcatcaatggttttgctttggcctggttggcaatacgagcgatggttgttccacgcactgacaacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgacttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgataaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaagcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagcatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtacctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgcataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgaaaaccacccatataggacatgggcttaccatggaagctatgtggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacgaccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaatacagtcaacatggtgcgcaggatcataggtgatgaagaaaagtacatggactacctatccacccaagttcgctacttgggtgaagaagggtctacacctggagtgctgtgagcaccaatcttaatgttgtcaggcctgctagtcagccacagcttggggaaagctgtgcagcctgtgacccctccaggagaagctgggtaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatcca

SEQ ID NO: 10 - DNA, 20 bp, artificial sequence
  synthetic primer PCR forward 1
tactcgagatgggcgcaaag

SEQ ID NO: 11 - DNA, 20 bp, artificial sequence
  synthetic primer-PCR forward 2
acctagcttctgggcgagtt

SEQ ID NO: 12 - DNA, 19 bp, artificial sequence
  synthetic primer-PCR forward 3
gcccaacacaaggtgaagc

SEQ ID NO: 13 - DNA, 21 bp, artificial sequence
  synthetic primer PCR reverse 1
ccagtgcttctttgttgttcc

SEQ ID NO: 14 - DNA, 20 bp, artificial sequence
  synthetic primer PCRreverse 2
ttgtgatggcaggttccgta

SEQ ID NO: 15 - DNA, 20 bp, artificial sequence
  synthetic primer PCR reverse 3
cgcggttagtgatggtgatg

SEQ ID NO: 16 - DNA, 16 bp, artificial sequence
  synthetic primer seq forward 1
gtaaaacgacggccag

SEQ ID NO: 17 - DNA, 20 bp, artificial sequence
  synthetic primer-seq forward 2
cccaagttgatgtcgtgttg

SEQ ID NO: 18 - DNA, 24 bp, ARTIFICIAL SEQUENCE
  synthetic primer-seq forward 3
tgaccaagtatatgactttttggc

SEQ ID NO: 19 - DNA, 20 bp, artificial sequence
  synthetic primer seq forward 4
gcagctctaatgcgctgtta

SEQ ID NO: 20 - DNA, 17 bp, artificial sequence
  synthetic primer seq rev 1
caggaaacagctatgac

SEQ ID NO: 21 - DNA, 22 bp, artificial sequence
  synthetic primer seq rev 2
aacttagattgaagggcgtgtc

SEQ ID NO: 22 - DNA, 20 bp, artificial sequence
  synthetic primer-seq rev 3
ccaccatttggggactctta

SEQ ID NO: 23 - DNA, 20 bp, artificial sequence
  synthetic primer-seq rev 4
ccatgatctgtatataacac

SEQ ID NO: 24 - protein, 22 aa, artificial sequence
  synthetic construct (M+POWV Natural SP)
MRSGVDWTWIFLTMALTMAMAT

SEQ ID NO: 25 - protein, 22 aa, artificial sequence
  synthetic construt (M+D6W POWV SP)
MRSGVWWTWIFLTMALTMAMAT

SEQ ID NO: 26 - protein, 22 aa, artificial sequence
  synthetic construct (M+D6K POWV SP)
MRSGVKWTWIFLTMALTMAMAT

SEQ ID NO: 27 - protein, 22 aa, artificial sequence
  synthetic construct (genus engineered POWV SP)
  MISC_FEATURE: X1 is M or absent
  misc_feature (1)..(1): Xaa can be any naturally occurring amino acid
  MISC_FEATURE (6)..(6): X6 is A, I, L, M, F, H, V, P, G, Y, W, R, or K
XRSGVXWTWIFLTMALTMAMAT

SEQ ID NO: 28 - protein, 3415 aa, Powassan Virus (strain LB)
MMTTSKGKGGGPPRRKLKVTANKSRPATSPMPKGFVLSRMLGILWHAVTGTARPPVLKMFWKTVPLRQAEAVLKKIKRVIGNLMQSLHMRGRRRSGVDWTWIFLTMALMTMAMATTIHRDREGYMVMRASGRDAASQVRVQNGTCVILATDMGEWCEDSITYSCVTIDQEEEPVDVDCFCRGVDRVKLEYGRCGRQAGSRGKRSVVIPTHAQKDMVGRGHAWLKGDNIRDHVTRVEGWMWKNKLLTAAIVALAWLMVDSWMARVTVILLALSLGPVYATRCTHLENRDFVTGTQGTTRVSLVLELGGCVTITAEGKPSIDVWLEDIFQESPAETREYCLHAKLTNTKVEARCPTTGPATLPEEHQANMVCKRDQSDRGWGNHCGFFGKGSIVACAKFECEEAKKAVGHVYDSTKITYVVKVEPHTGDYLAANETNSNRKSAQFTVASEKVILRLGDYGDVSLTCKVASGIDVAQTVVMSLDSSKDHLPSAWQVHRDWFEDLALPWKHKDNQDWNSVEKLVEFGPPHAVKMDVFNLGDQTAVLLKSLAGVPLASVEGQKYHLKSGHVTCDVGLEKLKLKGTTYSMCDKAKFKWKRVPVDSGHDTVVMEVSYTGSDKPCRIPVRAVAHGVPAVNVAMLITPNPTIETNGGGFIEMQLPPGDNIIYVGDLSQQWFQKGSTIGRMFEKTRRGLERLSVVGEHAWDFGSVGGVLSSVGKAIHTVLGGAFNTLFGGVGFIPKMLLGVALVWLGLNARNPTMSMTFLAVGALTLMMTMGVGADYGCAIDPERMEIRCGEGLVVWKEVSEWYDGYAYHPESPDTLAQALREAFERGVCGVVPQNRLEMAMWRSTAPELNLVLSEGEANLTIVVDKTDPADYRGGTPMVLKKTGKESKVSWKSWGKSILWSVPDSPRRMMMGVDGVGECPLYRRATGVFTVAEFGVGLRTKVFLDLRGEASKECDTGVMGAAVKNGKAIHTDQSMWMSSFRNDTGTYIHELILTDLRNCTWPASHTIDNDGVLDSHLFLPVTLAGPRSKYNRIPGYSEQVRGPWDQTPLRVVRDHCPGTSVRIDSHCDKRGASVRSTTESGKIIPEWCCRACELPPVTFRSGTDCWYAMEIRPVHSQGGLVRSMVVADNGALLSEGGVPGLVAVFVLMEFLLRRRPGSVTSILWGGILMLGLLVTGLVRVEEIVRYVIAVGVTFHLELGPETMVLVMLQAVFNMRTCYLMGFLVKRVITTREVVTVYFLLLVLEMGIPEMNFGHLWEWADALAMGLLIIKASAMEDRRGLGFLLAGLMTQRHLVAVHHGLMVFLTVALAVVGRNIYNGQKERKGLCFTVPLASLLGGSGSGLRMLALWECLGGRGRRSLSEPLTVVGVMLAMASGLLRHSSQEALLALSAGSFLILMLILGTRRLQLTAEWAGVVEWNPELVNEGGEVSLKVRQDAMGNLHLTEVEREERRLALWLVFGLLASAYHWSGILVTMGAWTVYELFSSTRRTDLVFSGQLPDQGEKRSFDIKEGVYRIYAPGLFWGYRQIGVGYGTKGVLHTMWHVTRGAALSVEGATSGPYWADVREDVVCYGGAWGLDKKWGGEVVQVHAFPPDSGHKIHQCQPGKLNLEGGRVLGAIPIDLPRGTSGSPIINAQGDVLGLYGNGLKSNDVYISSIAQGNVEKSRPEMPLAVQGGKWTSKGSITVLDMHPGSGKTHRVLPELIRECIDKRLRTVVLAPTRVVLKEMERALQGKRVKFHSAAVDNASSSSGAIVDVMCHATYVNRRLLPQGRQNWEVAIMDEAHWTDPHSIAARGHLYSLAKENRCALVLMTATPPGKSEAFPESKGAIVSEEKPIPEGEWRDGFDWITEFEGRTAWFVPSIAKGGAIARTLRQKGKSVICLNSKTFDKDYGRVHEEKPDFVVTTDISEMGANLDVNRVIDGRTNIKPEEIDGKVELIGTRRVTTASAAQRRGRVGRHEGRTDLYVYSGQCDDDDSSLVQWKEAQILLDNITTVRGPVATFYGPEQGKMLEVAGHFRLTEEKRKHFRHLLTNCDFTPWLAWHVAANTACVTDRKWTWEGPDENAIDGPGGELVTFRSPNGAERKLKPIWKDSRMFREGRDVADFIQYASGRRSAVDILTGLGGVPDLLRLRCTAAWDVVYTLLNETPGSRAMKMAERDAPEAMLTLLEVAVLGIATLGVVWCFIVRTSVSRMVLGTLVLAVALILLWLGGMDYGTMAGVALIFYLLLTVLQPEPGKQRSGEDNRLAFLLIGLGSVVGLVAANELGYLEQTKTDISGLFRREDQGGMVWDAWTNIDIQPARSWGTYVLIVSLFTPYMLHQLQTKIQRLVNSSVAAGTQAMRDLGGGTPFFGVAGHVVALGVTSLVGATPTSLALGVALAALHLAVVTSGLEAELTQRAHRAFFSAMVKNPMVDGEIINPIPDGDPKPALYERKMSLFLAIGLCIAAVALNRTAAAMTEAGAVAVAALGQLLRPEEESWWTMPMACGMAGLVRGSLWGLLPVLHRIWLRTQGARRGGAEGSTLGDIWKQRLNSCTKEEFFAYRRTGVMETNRDQARELLRRGETNMGLAVSRGCAKLAWLEERGYATLKGEVVDLGCGRGGWSYYAASRPSVMAVRAYTIGGKGHEAPRLVTSLGWNLIKFRSGMDVFSMATTRADTILCDIGESSPDPEKEGARSRRVILLMEQWKARNPDAAAVFKVLAPYRPEVLEALHRFQLQWGGGLVRVPFSRNSTHEMYYSTAVTGNLVNSVNVLSRKLLARFGETRGPIQVPEIDLGTGTRCVTLAEDKVKPRDVAERIGALREQYSESWHEDKEHPYRTWQYWGSYRTPATGSAASLINGVVKLLSWPWNAREDVTRMAMTDTTAFGQQRVFKEKVDTKAQEPQPGTRVIMRAVSDWLLEHLSRRAKVRMCTKDEFIAKVRSNAALGAWSDEQNKWSSAKEAVEDPEFWKLVDEERSRHLKGQCRHCVYNMMGKREKKLGEFGVAKGSRAIWYMWLGSRFLEFEVLGFLNEEHWASREVSGAGVEGTSLNYLGWLLRELGMKDGGKLYADDTAGWDTRITNADLEDEEQILRYMEGEHHVLAKTILEKAYHAKVVKVARPSPQGGCVMDVITRRDQRGSGQVVTYALNTITNMKVQLIRMMEGEGVIGPADSQDPRLKRVETWLKEHGVERLGRMLVSGDDCVVKPIDDRFGKALYFLNDMAKVRKDVGEWEPSMGFTEWEEVPFCSHHFHELVMKDGRSLIVPCRDQDELVGRARVSPGCGWSVRETACLSKAYGQMWLLNYFHRRDLRTLGFAICSAVPVSWVPMGRTTWSIHASGEWMTTEDMLRIWNKVWILDNPHMEDKQTVDEWRDIPYLPKTQDLVCSSLVGRKERAEWAKNIWGSVEKVRKLIGPEDYRDYLSSMDRHDLHWELKLESSII

SEQ ID NO: 29 - protein, 497 aa, powassan virus (strain LB)
TRCTHLENRDFVTGTQGTTRVSLVLELGGCVTITAEGKPSIDVWLEDIFQESPAETREYCLHAKLTNTKVEARCPTTGPATLPEEHQANMVCKRDQSDRGWGNHCGFFGKGSIVACAKFECEEAKKAVGHVYDSTKITYVVKVEPHTGDYLAANETNSNRKSAQFTVASEKVILRLGDYGDVSLTCKVASGIDVAQTVVMSLDSSKDHLPSAWQVHRDWFEDLALPWKHKDNQDWNSVEKLVEFGPPHAVKMDVFNLGDQTAVLLKSLAGVPLASVEGQKYHLKSGHVTCDVGLEKLKLKGTTYSMCDKAKFKWKRVPVDSGHDTVVMEVSYTGSDKPCRIPVRAVAHGVPAVNVAMLITPNPTIETNGGGFIEMQLPPGDNIIYVGDLSQQWFQKGSTIGRMFEKTRRGLERLSVVGEHAWDFGSVGGVLSSVGKAIHTVLGGAFNTLFGGVGFIPKMLLGVALVWLGLNARNPTMSMTFLAVGALTLMMTMGVGA

SEQ ID NO: 30 - protein, 163 aa, powassan virus (LB strain)
TIHRDREGYMVMRASGRDAASQVRVQNGTCVILATDMGEWCEDSITYSCVTIDQEEEPVDVDCFCRGVDRVKLEYGRCGRQAGSRGKRSVVIPTHAQKDMVGRGHAWLKGDNIRDHVTRVEGWMWKNKLLTAAIVALAWLMVDSWMARVTVILLALSLGPVYA

SEQ ID NO: 31 - DNA, 10389 bp, Powassan virus (strain LB)
caagagctgggagtggttatgatgaccacttctaaaggaaaggggggcggtccccctaggcgcaagcttaaagtgaccgcaaataagtcgcgaccagcaacgagcccaatgccaaagggcttcgtgctgtcgcgcatgctggggattctttggcacgccgtgacaggcacggccagacccccagtgctgaaaatgttctggaaaacggtaccactgcgccaggcggaggctgttctgaagaagataaagagagttatcgggaacttgatgcagagcctccacatgagagggcgtcgcaggtcaggtgtggactggacttggatttttttgacgatggcgttgatgaccatggccatggcaaccaccatccaccgggacagggaaggatacatggttatgcgggccagtggaagggacgctgcaagccaggtcagggtacaaaacggaacgtgcgtcatcctggcaacagacatgggagagtggtgtgaagattcaatcacctactcttgcgtcacgattgaccaggaggaagaacccgttgacgtggactgcttctgccgaggtgttgatagggttaagttagagtatggacgctgtggaaggcaagctggatctagggggaaaaggtctgtggtcattccaacacatgcacaaaaagacatggtcgggcgaggtcatgcatggcttaaaggtgacaatattcgagatcatgtcacccgagtcgagggctggatgtggaagaacaagcttctaactgccgccattgtggccttggcttggctcatggttgatagttggatggccagagtgactgtcatcctcttggcgttgagtctagggccagtgtacgccacgaggtgcacgcatcttgagaacagagattttgtgacaggaactcaagggaccaccagagtgtccctagttttggaacttggaggctgcgtgaccatcacagctgagggcaagccatccattgatgtatggctcgaagacatttttcaggaaagcccggctgaaaccagagaatactgcctgcacgccaaattgaccaacacaaaagtggaggctcgctgtccaaccactggaccggcgacacttccggaggagcatcaggctaatatggtgtgcaagagagaccaaagcgaccgtggatggggaaaccactgcgggttttttgggaagggcagtatagtggcttgtgcaaagtttgaatgcgaggaagcaaaaaaagctgtgggccacgtctatgactccacaaagatcacgtatgttgtcaaggttgagccccacacaggggattacttggctgcaaatgagaccaattcaaacaggaaatcagcacagtttacggtggcatccgagaaagtgatcctgcggctcggcgactatggagatgtgtcgctgacgtgtaaagtggcaagtgggattgatgtcgcccaaactgtggtgatgtcactcgacagcagcaaggaccacctgccttctgcatggcaagtgcaccgtgactggtttgaggacttggcgctgccctggaaacacaaggacaaccaagattggaacagtgtggagaaacttgtggaatttggaccaccacatgctgtgaaaatggatgttttcaatctgggggaccagacggctgtgctgctcaaatcactggcaggagttccgctggccagtgtggagggccagaaataccacctgaaaagcggccatgttacttgtgatgtgggactggaaaagctgaaactgaaaggcacaacctactccatgtgtgacaaagcaaagttcaaatggaagagagttcctgtggacagcggccatgacacagtagtcatggaggtatcatacacaggaagcgacaagccatgtcggatcccggtgcgggctgtggcacatggtgtcccagcggttaatgtagccatgctcataacccccaatccaaccattgaaacaaatggtggcggattcatagaaatgcagctgccaccaggggataacatcatctatgtgggagaccttagccagcagtggtttcagaaaggcagtaccattggtagaatgtttgaaaaaacccgcaggggattggaaaggctctctgtggttggagaacatgcatgggactttggctcagtaggcggggtactgtcttctgtggggaaggcaatccacacggtgctggggggagctttcaacaccctttttgggggggttggattcatccctaagatgctgctgggggttgctctggtctggttgggactaaatgccaggaatccaacgatgtccatgacgtttcttgctgtgggggctttgacactgatgatgacaatgggagttggggcagattatggatgtgccattgacccagaaaggatggaaatccgctgtggagaagggcttgttgtgtggaaagaggtgtcggaatggtatgacggctacgcctatcacccagaatcacctgacacgcttgcacaggctctcagagaggcctttgagagaggcgtttgtggggtcgtgccccaaaacagacttgaaatggccatgtggagaagcactgctccggaactcaacttggtgctgagtgagggagaagcaaacttgaccatcgtggtagacaaaacagatccggcagactataggggaggcactcccatggttctaaagaagactggaaaagagagcaaggtgtcctggaagagctggggcaagtcaatcttgtggagtgttcctgatagccccaggcgcatgatgatgggggttgacggagttggggagtgccccttgtataggagagcaacaggagtgttcacagtggcagagtttggagtgggcctacgcacgaaagtgttcttggatctgcgtggtgaagcttcaaaagagtgtgacacaggagtgatgggagcagccgtgaagaatggaaaagccatccacacggaccaaagtatgtggatgagctcctttcggaatgacacgggcacgtacatccatgagcttattctcactgatctcaggaactgcacatggccagcaagtcacactattgataatgatggagttttagactcccatcttttcttgccagtcacccttgctgggcccaggtcaaagtacaacaggatacctgggtacagtgaacaagtaagaggaccatgggaccagactcctctgagggtggtccgggaccattgccctggaacaagtgtgaggattgacagccattgtgacaagagaggagcttcagttagaagcaccacagaaagtggaaagatcattccagagtggtgctgccgggcctgtgagctaccaccggtgacattccgtagtggcacggattgttggtacgctatggaaatcaggccagtccattcgcagggcggcttggtgcgttccatggtagtggctgacaatggtgccttgcttagtgagggaggagttcctggtctagtggcagtgtttgtattgatggaattccttttgagaaggcgtccgggatctgtcacgtccattttgtggggaggcatactaatgctgggcttgctggtcacgggtctcgtgagagtggaggagatcgtgcgctatgtcatagcagttggggtcactttccacttggaattgggaccagagaccatggttcttgtgatgctgcaggcggtcttcaacatgcgcacatgttatctcatggggtttcttgtcaagagggtgatcaccacacgtgaggtggtgactgtttacttcttgctattggtccttgagatggggataccggagatgaacttcggacacctctgggaatgggctgatgcactggccatggggttgctcataatcaaggccagtgcgatggaagatcgcagaggtttgggattcttgctggctgggctcatgacacaaagacacctagtcgctgtccatcatggtttgatggtatttctcactgtggccttggcggtagtgggacggaatatctacaatggacaaaaagagaggaaaggactgtgtttcacagtccccctggcctcactacttggtggctccggctccggattgcgcatgctggccttgtgggaatgtcttgggggtcgtggcagacggagcctcagtgagccccttactgtggtgggggtcatgctggccatggccagtggactactacgacattcatcccaagaggctcttttggccctttctgcaggatcctttctcattttgatgctgatcctggggacccgtagactccagctaacggcggaatgggccggagtggtggagtggaaccctgagttggtcaatgagggaggtgaggtaagcttgaaggtcagacaggatgccatggggaacttgcacttgacagaggtggaacgggaggagcgtcgtttggccttatggcttgtgttcggcctactggcatcggcttatcattggtccggaatattggtcacaatgggggcttggacagtttatgagctcttctcatccacgcggagaactgacctggttttttcagggcagttgcctgaccaaggggaaaaacgttcctttgacatcaaggaaggcgtataccgcatatacgcccctgggctcttctggggttaccgccagataggtgtgggatatggaacgaaaggagtcttacacaccatgtggcatgtgacaagaggagccgccctcagcgtggagggggccacatcaggaccatactgggcggatgtgcgtgaagacgtggtctgctatgggggggcctggggcttggacaaaaaatggggaggcgaagtcgttcaggtacacgcattccctccggacagtggacacaaaatccatcaatgccagccaggaaagctgaaccttgagggggggagagttctgggggccatcccgatagacctcccacgtggtacttcaggtagcccaatcatcaatgcacagggagatgtgctgggtctgtacggcaatggtctgaaatcaaatgatgtttacatcagtagcattgctcagggcaatgttgaaaagagtcgccctgagatgccattggcagtccagggtggcaaatggacatctaagggcagcatcacagttttggatatgcatcccgggtcgggaaagacacacagagtccttcccgaactgattcgtgagtgcattgacaaaaggttgaggactgttgtattggcgccaacacgggtggtcctgaaggaaatggaaagggccttgcaggggaaacgtgtcaagtttcattcggcagcagttgacaatgcttcttcatcatcgggagctattgttgatgtcatgtgtcatgccacctacgtcaaccgccggctcttgccacaagggagacagaactgggaggtggccataatggatgaggcacattggaccgacccacacagcatagctgcccgaggccatctttactccttggccaaggagaacagatgtgcactcgtgctgatgactgctaccccacctggcaagagcgaggcgttccctgaatccaaaggagccatagtgagtgaggaaaaacccattcccgaaggagagtggagagatggctttgattggataacagaatttgagggtcgaacagcctggtttgtgccatcgatagccaagggaggcgccatagcacggacactgagacagaaagggaaaagcgtgatctgtctgaacagtaagacctttgacaaggattatggcagagtccatgaggaaaaacctgacttcgtcgtgacaactgacatctcagaaatgggggccaatctagatgttaatcgggtcatagatggtagaactaacatcaaacccgaggagatagatggaaaggtggaattgattggcacgagacgagtgacgacagcatcagctgcgcaacgcagagggcgagttggaaggcatgaaggacgcacggacctctatgtgtattctgggcagtgtgatgatgatgacagctccttagtgcagtggaaagaagcgcagattctgcttgacaacattactacagtgagaggaccagttgccactttttatggtccagagcaagggaaaatgctcgaggtggcggggcacttccgcttgacggaggaaaaaagaaagcacttccggcaccttctgacaaactgtgacttcacgccctggctcgcatggcacgtggctgcaaacactgcctgtgtgacggaccggaaatggacctgggaaggtcccgacgagaatgccattgatggtccaggcggcgaactggtcacgttcagaagccccaatggagcggaacggaagctgaagccaatctggaaggacagtagaatgttccgggaggggagagatgtggcagacttcatccagtacgcttccggtcgtaggagtgccgtggacatcctgactggacttggtggagtcccagacctgctgcggttgcgctgcacagcggcgtgggatgttgtctacacactgctcaatgagaccccagggagtcgcgccatgaaaatggcggagagagatgctcctgaggcgatgctgacacttttggaggtagcggttctgggcatcgccacactgggggtggtttggtgtttcattgtccggacgtcagtcagcagaatggttctgggcaccctggtgttggcagtggctctcatcctgttgtggttgggaggaatggactatggaaccatggcaggagtggctctcatcttctatttgctcctcacggtactgcaaccagaaccgggaaagcagcgtagtggagaggataaccgactggccttcctgttgattggactggggagcgtggtgggccttgtggcagccaatgagctgggctatctggaacaaacgaaaactgacatttcaggactctttagacgtgaggaccagggagggatggtttgggacgcatggacgaacatcgacatacagcctgcccgatcctggggaacttatgtgctcattgtctccctctttactccatatatgctccatcagctacaaacaaaaatccagaggctggtgaacagctcagttgcagcgggaacacaggccatgagagaccttgggggaggcaccccattctttggtgtagctggacatgtcgtggcactcggtgtgacctctttggtaggagcgacgcccacctcccttgcattgggtgttgccttggctgccctccatttggccgtggtcacatctgggctggaggctgaactgacccagagggcgcaccgggcatttttctcagccatggtaaagaatccaatggtggatggagagatcatcaaccccattccggatggagacccaaaaccagctttgtatgagaggaagatgagtttgtttctcgccatcggcctgtgcatagcggccgtagccttgaacaggactgccgcggcgatgacagaagctggcgcagtggctgtggcggctttgggacagttgctgaggcccgaagaggagagctggtggaccatgcccatggcttgcggaatggctggactggtgcggggaagcctatggggcttactgccagttcttcaccgcatctggctcagaacccagggagctagaaggggtggagcagagggaagcacccttggtgacatttggaagcagcggctgaactcatgcaccaaagaagaattttttgcctacaggcgcacaggggtcatggagacaaaccgagaccaagctcgtgaattgcttcgccgaggtgagactaacatggggttggcagtatcgcgaggctgtgccaaactagcttggctagaggaaagggggtacgcgacactcaaaggagaggttgtggaccttggttgtggaagggggggctggtcatattatgccgcctcaagaccatccgtcatggctgtgagagcttacaccattggagggaagggccatgaagcaccacgccttgtgacgagtcttggatggaatctcataaagttcaggagtgggatggatgtgttctcaatggcgaccacaagagcagacacaatcttgtgtgacattggggagagcagtccagatccggagaaggaaggggctcggagccgaagagtgattcttttgatggaacaatggaaagccagaaacccagacgccgcggcagtcttcaaggtgttggccccgtataggccagaggtgctcgaggctctgcacagatttcagctacaatgggggggggggcttgtgagagtccctttctcacggaattccacccatgaaatgtactattccacggccgtcacaggaaatcttgttaattcagtcaatgtgctctcgcggaagctcctggctagatttggggagacgagaggacccatccaggtgcctgagatcgacctgggaactggaaccaggtgtgtgacgctagctgaggacaaagtgaagcccagggacgtggcggagcgcattggagccttgagggaacagtactcagagtcgtggcacgaagacaaagaacatccctaccggacatggcaatactggggcagctatcgcactccggctaccggatcagcggcatccctgatcaacggagtggtgaagctgctaagctggccttggaatgcacgggaggatgttaccagaatggccatgacagacacaacggcctttgggcagcagcgagttttcaaagaaaaggttgacacaaaggcccaagagccgcagccaggcaccagagtgatcatgagagctgtgagtgactggctgttggagcatctaagccgccgagccaaagtgaggatgtgtaccaaagatgagttcatagcgaaggtgaggtccaacgctgctctaggtgcatggtcggatgaacagaataagtggagctcggcaaaagaggcagtggaagatccagaattttggaaattggtggatgaagagcgctccagacacttgaaaggtcagtgccgacattgtgtgtacaacatgatgggaaagagagagaagaagctaggtgagtttggagttgcaaaaggcagcagggccatctggtacatgtggcttggaagccgcttccttgagtttgaggtccttgggtttcttaatgaggagcattgggcttccagagaggtgagtggagctggggtggaaggaactagtctcaactatctgggctggctgctaagagaacttggaatgaaagacggtggcaaactgtacgccgacgataccgctggctgggacactcggatcaccaatgcagatttggaagatgaagagcagatccttcggtacatggaaggagaacaccatgtgctggcaaaaacaatacttgaaaaggcttaccatgccaaagtggtcaaggttgccaggccctcaccccaagggggatgtgtgatggacgtgatcacaaggagagaccagaggggatcaggacaggtcgtcacatacgccctcaacaccatcacaaacatgaaagtccaactcatccgcatgatggaaggagaaggagtcataggaccagctgactcacaggaccctcgtctgaaaagggtagaaacctggttgaaggagcacggagttgaaagactagggaggatgcttgtgagtggagatgattgtgtcgtcaagcccatcgatgacaggttcggaaaggcgctgtacttcctgaatgacatggccaaggttcgtaaggatgtgggagagtgggagccctcgatgggcttcacagagtgggaggaggtccctttctgttcacatcacttccatgaacttgtgatgaaggatggacgctctctcatagtgccatgcagagaccaagatgagctggttggcagggcaagggtgtcaccaggatgtggatggagtgtgcgagagacggcctgcctgtccaaggcgtacgggcagatgtggcttctcaactactttcatcgcagagaccttcgcacccttggtttcgccatatgttctgctgttcccgtgagttgggttcccatgggtcgcaccacttggagcatccacgcgagtggtgaatggatgacaacagaagacatgctgcgcatctggaacaaagtctggattttggacaaccctcacatggaagataagcagacagtggatgagtggagagacatcccctatctgccaaaaacacaggacctggtatgttcctcacttgttgggagaaaggagagggcagaatgggctaagaacatctggggctcagtggagaaggtcaggaagctcattggaccagaagactacagagactacttatcaagcatggacagacatgacctgcactgggagctcaagctagagagctcgataatctaaactagcatgactgaacagtcaaaagaaccctaacacaggggatggtgtggcagcgcacaacgacatcgtgacgggagtgggtcgcccccgacgcaccatcctcttgggaaaaattttcgtgag

SEQ ID NO: 32 - DNA, 4371 bp, artificial sequence
  synthetic construct (vIND-ZIKV region with E alone)
tgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaagaaaaacccgggttacttgtaaagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatactagttatttatagcatagaaaaaaacaaaatgaaattctactatatagaaaaaccatggttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcaaagaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatgctagctatttatattccaaaaaaaaaaaataaaatttcaatttttaactgcagttaaaaattgaaattttattttttttttttggaatataaatactcgagatgatcaggtgcataggagtcagcaatagggactttgtggaaggtatgtcaggtgggacttgggttgatgttgtcttggaacatgggggttgtgtcaccgtaatggcacaggacaaaccaacagtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcttaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggactcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccaccgcggtttctgctcatcatcatcaccatcactaaccgcggtttctgcttaatttttgtggatcctgtacaagtaagcatgcatatatgctcatatatttatagaagatatcacatatctaaatatccctatcagtgatagagacggccgatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaatt

SEQ ID NO: 33 - DNA, 4929 bp, artificial sequence
  synthetic construct (vIND-ZIKV region with wt signal protein)
tgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaagaaaaacccgggttacttgtaaagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatactagttatttatagcatagaaaaaaacaaaatgaaattctactatatagaaaaaccatggttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcaaagaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatgctagctatttatattccaaaaaaaaaaaataaaatttcaatttttaactgcagttaaaaattgaaattttattttttttttttggaatataaatactcgagatgggcgcagatactagtgtcggaatcgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgatgtcgattgttggtgcaacacgacatcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatgggggttgtgtcaccgtaatggcacaggacaaaccaacagtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcttaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggactcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccaccgcggtttctgctcatcatcatcaccatcactaaccgcggtttctgcttaatttttgtggatcctgtacaagtaagcatgcatatatgctcatatatttatagaagatatcacatatctaaatatccctatcagtgatagagacggccgatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaatt

SEQ ID NO: 34 - DNA, 4929 bp, artificial sequence
  synthetic construct (vIND-ZIKV region with D4W SP sequence)
tgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaagaaaaacccgggttacttgtaaagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatactagttatttatagcatagaaaaaaacaaaatgaaattctactatatagaaaaaccatggttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcaaagaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatgctagctatttatattccaaaaaaaaaaaataaaatttcaatttttaactgcagttaaaaattgaaattttattttttttttttggaatataaatactcgagatgggcgcatggactagtgtcggaatcgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgatgtcgattgttggtgcaacacgacatcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatgggggttgtgtcaccgtaatggcacaggacaaaccaacagtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcttaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggactcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccaccgcggtttctgctcatcatcatcaccatcactaaccgcggtttctgcttaatttttgtggatcctgtacaagtaagcatgcatatatgctcatatatttatagaagatatcacatatctaaatatccctatcagtgatagagacggccgatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaatt

SEQ ID NO: 35 - DNA, 4929 bp, artificial sequence
  synthetic construct (vIND-ZIKV regions with D4K SP)
tgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaagaaaaacccgggttacttgtaaagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatactagttatttatagcatagaaaaaaacaaaatgaaattctactatatagaaaaaccatggttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcaaagaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatgctagctatttatattccaaaaaaaaaaaataaaatttcaatttttaactgcagttaaaaattgaaattttattttttttttttggaatataaatactcgagatgggcgcaaagactagtgtcggaatcgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgatgtcgattgttggtgcaacacgacatcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatgggggttgtgtcaccgtaatggcacaggacaaaccaacagtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcttaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggactcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccaccgcggtttctgctcatcatcatcaccatcactaaccgcggtttctgcttaatttttgtggatcctgtacaagtaagcatgcatatatgctcatatatttatagaagatatcacatatctaaatatccctatcagtgatagagacggccgatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaatt

SEQ ID NO: 36 - DNA, 4941 bp, artificial sequence
  synthetic construct (vIND-ZIKV region JEV construct)
tgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaagaaaaacccgggttacttgtaaagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatactagttatttatagcatagaaaaaaacaaaatgaaattctactatatagaaaaaccatggttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcaaagaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatgctagctatttatattccaaaaaaaaaaaataaaatttcaatttttaactgcagttaaaaattgaaattttattttttttttttggaatataaatactcgagatgggtggaaacgaaggttcaattatgtggcttgcaagtttggctgtagttattgcgtgcgcaggagcggcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgatgtcgattgttggtgcaacacgacatcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatgggggttgtgtcaccgtaatggcacaggacaaaccaacagtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcttaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggactcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccaccgcggtttctgctcatcatcatcaccatcactaaccgcggtttctgcttaatttttgtggatcctgtacaagtaagcatgcatatatgctcatatatttatagaagatatcacatatctaaatatccctatcagtgatagagacggccgatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaatt

SEQ ID NO: 37 - DNA, 4829 bp, artificial sequene
  synthetic construct (vIND-POWV natural SP)
tgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaagaaaaacccgggttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcaaagaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatgctagctatttatattccaaaaaaaaaaaataaaatttcaatttttaactgcagttaaaaattgaaattttattttttttttttggaatataaataatgaggtcaggtgtggactggacttggattttcttgacgatggcgttgatgaccatggccatggcaaccaccatccaccgggacagggaaggatacatggttatgcgggccagtggaagggacgctgcaagccaggtcagggtacaaaacggaacgtgcgtcatcctggcaacagacatgggagagtggtgtgaagattcaatcacctactcttgcgtcacgattgaccaggaggaagaacccgttgacgtggactgcttctgccgaggtgttgatagggttaagttagagtatggacgctgtggaaggcaagctggatctagggggaaaaggtctgtggtcattccaacacatgcacaaaaagacatggtcgggcgaggtcatgcatggcttaaaggtgacaatattcgagatcatgtcacccgagtcgagggctggatgtggaagaacaagcttctaactgccgccattgtggccttggcttggctcatggttgatagttggatggccagagtgactgtcatcctcttggcgttgagtctagggccagtgtacgccacgaggtgcacgcatcttgagaacagagattttgtgacaggaactcaagggaccaccagagtgtcactagttttggaacttggaggctgcgtgaccatcacagctgagggcaagccatccattgatgtatggctcgaagacatttttcaggaaagcccggctgaaaccagagaatactgcctgcacgccaaattgaccaacacaaaagtggaggctcgctgtccaaccactggaccggcgacacttccggaggagcatcaggctaatatggtgtgcaagagagaccaaagcgaccgtggatggggaaaccactgcgggttttttgggaagggcagtatagtggcttgtgcaaagtttgaatgcgaggaagcaaaaaaagctgtgggccacgtctatgactccacaaagatcacgtatgttgtcaaggttgagccccacacaggggattacttggctgcaaatgagaccaattcaaacaggaaatcagcacagtttacggtggcatccgagaaagtgatcctgcggctcggcgactatggagatgtgtcgctgacgtgtaaagtggcaagtgggattgatgtcgcccaaactgtggtgatgtcactcgacagcagcaaggaccacctgccttctgcatggcaagtgcaccgtgactggtttgaggacttggcgctgccctggaaacacaaggacaaccaagattggaacagtgtggagaaacttgtggaatttggaccaccacatgctgtgaaaatggatgttttcaatctgggggaccagacggctgtgctgctcaaatcactggcaggagttccgctggccagtgtggagggccagaaataccacctgaaaagcggccatgttacttgtgatgtgggactggaaaagctgaaactgaaaggcacaacctactccatgtgtgacaaagcaaagttcaaatggaagagagttcctgtggacagcggccatgacacagtagtcatggaggtatcatacacaggaagcgacaagccatgtcggatcccggtgcgggctgtggcacatggtgtcccagcggttaatgtagccatgctcataacccccaatccaaccattgaaacaaatggtggcggattcatagaaatgcagctgccaccaggggataacatcatctatgtgggagaccttagccagcagtggtttcagaaaggcagtaccattggtagaatgtttgaaaaaacccgcaggggattggaaaggctctctgtggttggagaacatgcatgggactttggctcagtaggcggggtactgtcttctgtggggaaggcaatccacacggtgctggggggagctttcaacaccctttttgggggggttggattcatccctaagatgctgctgggggttgctctggtctggttgggactaaatgccaggaatccaacgatgtccatgacgtttcttgctgtgggggctttgacactgatgatgacaatgggagttggggcataaaattggatcagcttttttttttttttttttggcatataaataaggtcgaagcttggtaccaaaaattgaaaaactattctaatttattgcacatggcctcctccgaggacgtcatcaaagagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctccaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtaggcatgcatatatgctcatatatttatagaagatatcacatatctaaatatccctatcagtgatagagacggccgatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaatt

SEQ ID NO: 38 - DNA, 4829 bp, artificial sequence
  synthetic construct (vIND-POWV D6W SP)
tgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaagaaaaacccgggttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcaaagaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatgctagctatttatattccaaaaaaaaaaaataaaatttcaatttttaactgcagttaaaaattgaaattttattttttttttttggaatataaataatgaggtcaggtgtgtggtggacttggattttcttgacgatggcgttgatgaccatggccatggcaaccaccatccaccgggacagggaaggatacatggttatgcgggccagtggaagggacgctgcaagccaggtcagggtacaaaacggaacgtgcgtcatcctggcaacagacatgggagagtggtgtgaagattcaatcacctactcttgcgtcacgattgaccaggaggaagaacccgttgacgtggactgcttctgccgaggtgttgatagggttaagttagagtatggacgctgtggaaggcaagctggatctagggggaaaaggtctgtggtcattccaacacatgcacaaaaagacatggtcgggcgaggtcatgcatggcttaaaggtgacaatattcgagatcatgtcacccgagtcgagggctggatgtggaagaacaagcttctaactgccgccattgtggccttggcttggctcatggttgatagttggatggccagagtgactgtcatcctcttggcgttgagtctagggccagtgtacgccacgaggtgcacgcatcttgagaacagagattttgtgacaggaactcaagggaccaccagagtgtcactagttttggaacttggaggctgcgtgaccatcacagctgagggcaagccatccattgatgtatggctcgaagacatttttcaggaaagcccggctgaaaccagagaatactgcctgcacgccaaattgaccaacacaaaagtggaggctcgctgtccaaccactggaccggcgacacttccggaggagcatcaggctaatatggtgtgcaagagagaccaaagcgaccgtggatggggaaaccactgcgggttttttgggaagggcagtatagtggcttgtgcaaagtttgaatgcgaggaagcaaaaaaagctgtgggccacgtctatgactccacaaagatcacgtatgttgtcaaggttgagccccacacaggggattacttggctgcaaatgagaccaattcaaacaggaaatcagcacagtttacggtggcatccgagaaagtgatcctgcggctcggcgactatggagatgtgtcgctgacgtgtaaagtggcaagtgggattgatgtcgcccaaactgtggtgatgtcactcgacagcagcaaggaccacctgccttctgcatggcaagtgcaccgtgactggtttgaggacttggcgctgccctggaaacacaaggacaaccaagattggaacagtgtggagaaacttgtggaatttggaccaccacatgctgtgaaaatggatgttttcaatctgggggaccagacggctgtgctgctcaaatcactggcaggagttccgctggccagtgtggagggccagaaataccacctgaaaagcggccatgttacttgtgatgtgggactggaaaagctgaaactgaaaggcacaacctactccatgtgtgacaaagcaaagttcaaatggaagagagttcctgtggacagcggccatgacacagtagtcatggaggtatcatacacaggaagcgacaagccatgtcggatcccggtgcgggctgtggcacatggtgtcccagcggttaatgtagccatgctcataacccccaatccaaccattgaaacaaatggtggcggattcatagaaatgcagctgccaccaggggataacatcatctatgtgggagaccttagccagcagtggtttcagaaaggcagtaccattggtagaatgtttgaaaaaacccgcaggggattggaaaggctctctgtggttggagaacatgcatgggactttggctcagtaggcggggtactgtcttctgtggggaaggcaatccacacggtgctggggggagctttcaacaccctttttgggggggttggattcatccctaagatgctgctgggggttgctctggtctggttgggactaaatgccaggaatccaacgatgtccatgacgtttcttgctgtgggggctttgacactgatgatgacaatgggagttggggcataaaattggatcagcttttttttttttttttttggcatataaataaggtcgaagcttggtaccaaaaattgaaaaactattctaatttattgcacatggcctcctccgaggacgtcatcaaagagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctccaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtaggcatgcatatatgctcatatatttatagaagatatcacatatctaaatatccctatcagtgatagagacggccgatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaatt

SEQ ID NO: 39 - DNA, 4829 bp, artificial sequence
  synthetic construct (vIND-POWV D6K SP)
tgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaagaaaaacccgggttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcaaagaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatgctagctatttatattccaaaaaaaaaaaataaaatttcaatttttaactgcagttaaaaattgaaattttattttttttttttggaatataaataatgagatcaggcgtcaagtggacatggactttcttggtaatggctttgatggctatggccatggcgacctccatccatcgggataaagagggatatatggtcatgcgagctagcggaagagatgccgcaagccaagtcagagttcaaaacggaacatgcgtcattttggcaacggacatgggagaatggtgtgaagattcaataacctactcctgtgttaccattgaccaggaagaagaacctgttgatgtggattgtttttgccgaggtgttgacagagtcaagttagagtacggacgctgtggaagacaggctggattcagggggagaaggtctgtggtcatcccgacacatgcacaaaaggacatggttgggcgaggacacgcgtggctcaagggtgataacatccgagatcatgttacccgcgtcgagggctggatgtggaaaaacaagctcttgacagttgctgtcgtggctttggcttggctcatgctggatagttggatggctagggtgactgtgatccttctggcgctgagccttgggcctgtatacgccacgagatgcacacaccttgagaacagggactttgtgacaggagtacagggaaccactagagtatccctggttctggagcttggaggatgtgtgaccattactgctgaaggcaagccatcaattgatgtatggctggaggacatttttcaagaaagtcctgctgaaaccagagaatactgtttacacgccaagctctccaacacgaaagtagaggctcgttgccccaccaccgggccagctacgcttccagaggaacaccaggccaacatggtgtgcaagagagaccagagtgaccgtggatggggaaaccactgcggattttttgggaaaggaagcatagtggcctgtgccaagtttgaatgcgaggaagcaaaaaaggcagtaggtcatgtttatgactcaacaaaaatcacgtacgttgtcaaggttgagccccacacgggggactaccaagctgcaaatgaaaccaacgagaacaggaaaacggcccagttcacggtggcatctgagaaggtgatcttaaatctaggagactatggagatgtatcattgacatgtaaggtggcaagtggaattgatgtcgcccagactgtggtgatgtcgcttggcagcagcaaagatcacctgccttcggcatggcaggtgcatcgtgactggttcgaagacctagccctgccttggaaacacaaagacaatcaggactggaacagtgtggagaaacttgtggagtttgggccgcctcatgcagtgaaaatggatatcttcaatctgggggatcagactgctgttttgctcaaatcactggctggagtcccactggcaagcgtggacaaccagaaatatcacctgaaaagtggtcatgtcacctgtgatgtgggacttgaaaagctgaagctgaaaggcacaacttactccatgtgtgacaaaacaaagttcaagtggaaaagagttcctgtggacagtggccatgacacagtggtcatggaggtgtcttacacgggaagcgacaagccctgccggattccggtacgagcagtggcacatggtgtcccgacaattaatgtggccatgctcattacccccaacccaactattgaaacaagtggaggaggatttatagagatgcagttgccaccgggggacaacattatttatgtgggggacctaagtcagcagtggttccagaaaggcagtaccataggtagaatgttcgaaaagacccgtaagggtttggaaagattttcagtggttggagaacatgcatgggactttggttcagtgggcggaatcctgtcgtcagtggggaaagccgttcacacagtgttgggaggggcgttcaacaccctttttgggggagttgggttcatccctaagatgctgttgggagtcgccttggtgtggttggggctcaacgctaggaatccaacaatgtccatgacttttcttgccgtgggggttttgaccctgatgatgacaatgggagtcggagcataaaattggatcagcttttttttttttttttttggcatataaataaggtcgaagcttggtaccaaaaattgaaaaactattctaatttattgcacatggcctcctccgaggacgtcatcaaagagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctccaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtaggcatgcatatatgctcatatatttatagaagatatcacatatctaaatatccctatcagtgatagagacggccgatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaatt

SEQ ID NO: 40 - DNA, 4829 bp, artificial sequence
  synthetic construct (vIND-POWV JEV SP)
tgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaagaaaaacccgggttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcaaagaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatgctagctatttatattccaaaaaaaaaaaataaaatttcaatttttaactgcagttaaaaattgaaattttattttttttttttggaatataaataatgggaggaaatgaaggctcaatcatgtggctcgcgagcttggcagttgtcatagcttgtgcaggagccaccatccaccgggacagggaaggatacatggttatgcgggccagtggaagggacgctgcaagccaggtcagggtacaaaacggaacgtgcgtcatcctggcaacagacatgggagagtggtgtgaagattcaatcacctactcttgcgtcacgattgaccaggaggaagaacccgttgacgtggactgcttctgccgaggtgttgatagggttaagttagagtatggacgctgtggaaggcaagctggatctagggggaaaaggtctgtggtcattccaacacatgcacaaaaagacatggtcgggcgaggtcatgcatggcttaaaggtgacaatattcgagatcatgtcacccgagtcgagggctggatgtggaagaacaagcttctaactgccgccattgtggccttggcttggctcatggttgatagttggatggccagagtgactgtcatcctcttggcgttgagtctagggccagtgtacgccacgaggtgcacgcatcttgagaacagagattttgtgacaggaactcaagggaccaccagagtgtcactagttttggaacttggaggctgcgtgaccatcacagctgagggcaagccatccattgatgtatggctcgaagacatttttcaggaaagcccggctgaaaccagagaatactgcctgcacgccaaattgaccaacacaaaagtggaggctcgctgtccaaccactggaccggcgacacttccggaggagcatcaggctaatatggtgtgcaagagagaccaaagcgaccgtggatggggaaaccactgcgggttttttgggaagggcagtatagtggcttgtgcaaagtttgaatgcgaggaagcaaaaaaagctgtgggccacgtctatgactccacaaagatcacgtatgttgtcaaggttgagccccacacaggggattacttggctgcaaatgagaccaattcaaacaggaaatcagcacagtttacggtggcatccgagaaagtgatcctgcggctcggcgactatggagatgtgtcgctgacgtgtaaagtggcaagtgggattgatgtcgcccaaactgtggtgatgtcactcgacagcagcaaggaccacctgccttctgcatggcaagtgcaccgtgactggtttgaggacttggcgctgccctggaaacacaaggacaaccaagattggaacagtgtggagaaacttgtggaatttggaccaccacatgctgtgaaaatggatgttttcaatctgggggaccagacggctgtgctgctcaaatcactggcaggagttccgctggccagtgtggagggccagaaataccacctgaaaagcggccatgttacttgtgatgtgggactggaaaagctgaaactgaaaggcacaacctactccatgtgtgacaaagcaaagttcaaatggaagagagttcctgtggacagcggccatgacacagtagtcatggaggtatcatacacaggaagcgacaagccatgtcggatcccggtgcgggctgtggcacatggtgtcccagcggttaatgtagccatgctcataacccccaatccaaccattgaaacaaatggtggcggattcatagaaatgcagctgccaccaggggataacatcatctatgtgggagaccttagccagcagtggtttcagaaaggcagtaccattggtagaatgtttgaaaaaacccgcaggggattggaaaggctctctgtggttggagaacatgcatgggactttggctcagtaggcggggtactgtcttctgtggggaaggcaatccacacggtgctggggggagctttcaacaccctttttgggggggttggattcatccctaagatgctgctgggggttgctctggtctggttgggactaaatgccaggaatccaacgatgtccatgacgtttcttgctgtgggggctttgacactgatgatgacaatgggagttggggcataaaattggatcagcttttttttttttttttttggcatataaataaggtcgaagcttggtaccaaaaattgaaaaactattctaatttattgcacatggcctcctccgaggacgtcatcaaagagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctccaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtaggcatgcatatatgctcatatatttatagaagatatcacatatctaaatatccctatcagtgatagagacggccgatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaatt